(19) United States
(12) Patent Application Publication
(10) Pub. No.: US 2012/0214241 A1
(43) Pub. Date: Aug. 23, 2012

(54) ZINC FINGER NUCLEASE MODIFICATION OF LEUCINE RICH REPEAT KINASE 2 (LRRK2) MUTANT FIBROBLASTS AND IPSCS

(76) Inventors: Josee LAGANIERE, El Cerrito, CA (US); J. William Langston, Los Altos Hills, CA (US); Birgitt Schüle, Menlo Park, CA (US); H. Steve Zhang, Richmond, CA (US)

(21) Appl. No.: 13/327,322

(22) Filed: Dec. 15, 2011

Related U.S. Application Data
(60) Provisional application No. 61/426,462, filed on Dec. 22, 2010.
(60) Provisional application No. 61/520,235, filed on Jun. 6, 2011.

(51) Int. Cl. C12N 15/87; C12N 5/10

(52) U.S. Cl. 435/462; 435/325

(57) ABSTRACT

Disclosed herein are methods and compositions for alteration of an LRRK2 gene in a fibroblast or iPSC.

### CROSS-REFERENCE TO RELATED APPLICATIONS

The present application claims the benefit of U.S. Provisional Application Nos. 61/426,462 filed Dec. 22, 2010 and 61/520,235 filed Jun. 6, 2011, the disclosures of which are hereby incorporated by reference in their entireties.

### TECHNICAL FIELD

The present disclosure is in the fields of genome editing.

### BACKGROUND

Parkinson's disease (PD) is a neurodegenerative disease that afflicts approximately 4-6 million people worldwide. In the United States, approximately one to two hundred people per 100,000 have PD. Interestingly, the prevalence among Amish people is approximately 970 per 100,000 although the basis for this high rate, be it genetic or environmental, is not known. The prevalence of PD increases in the older population, with approximately 4% of people over the age of 80 suffering from this disease (Davie (2008) Brit Med Bull 86(1) p. 109), although 10% of patients are under 40 years of age (Kumari (2009) FEBS J. 276(22) p. 6455).

Typically a patient diagnosed with PD is identified by several hallmark physical behaviors: bradykinesia, rigidity and resting tremor. Often these physical symptoms are asymmetric. Within the brain, PD is characterized by a progressive and profound loss of neuromelanin-containing dopaminergic neurons in the substantia nigra pars compacta with the presence of eosinophillic, intracytoplasmic and proteinaceous inclusions termed Lewy bodies in the surviving neurons (Davie, ibid and Kumari, ibid). By the time of death, this region will have lost 50-70% of its neurons as compared to an individual without PD.

It appears that many factors can play a role in disease onset and/or progression of PD. For example, genetic mutations in the leucine rich repeat kinase 2 gene (LRRK2, also known as PARKS) has been identified to be involved in both familial and sporatic forms of PD. In fact, studies suggest that LRRK2 mutations may be responsible for between 5 and 13% of familial PD, and from 1 to 5% of sporadic PD. The protein itself is a large (>280 kD) multidomain protein containing the following known domains: armadillo (ARM), ankryn (ANK), LRR, Ras of complex proteins (ROC), C-terminal of ROC (COR), mitogen-activated protein kinase kinase kinase and WD40. Thus, LRRK2 contains several protein-protein interactive domains (ARM/ANK, LRR and WD40) suggesting that LRRK2 plays a role in protein complex formation (Kumari, ibid). Several clusters of mutations have been identified which fall across its length of the gene, with the majority of pathological mutations clustering in the enzymatic domains of the protein.

Specifically, the LRRK2 mutation G2019S has been suggested to play an important role in PD in some ethnicities. Although prevalence among Asians, South Africans and some Europeans (of Polish, Greek and German descent) is quite low, among Ashkenazi Jews and North African Arabs, the prevalence is from 37-40%. In the U.S. and the rest of Europe, G2019S accounts for approximately 1-7% of familial PD and 1-3% of sporadic PD in Caucasians (Kumari, ibid). The mutation is autosomal dominant and the lifetime penetrance for the mutation has been estimated at 31.8%. The SNP responsible for this missense mutation in patients is annotated as rs34637584 in the human genome, and is a G to A substitution at the genomic level (6055G>A). This LRRK2 mutation can be referred to either as G2019S or 6055G>A. The G2019S mutation has been shown to increase LRRK2 kinase activity, and is found in the within the activation domain of the protein (Luzon-Toro, (2007) Hum Mol Genet 16(17) p. 2031).

LRRK2 has also been shown to play a role some types of cancers. Although epidemiological evidence generally supports the idea that patients with PD are protected from cancer, it appears that for patients carrying certain LRRK2 mutations, there is an associated increase risk of melanoma as well as some renal and thyroid cancers (Looyenga et al (2011) Proc Nat'l Acad Sci USA. 108(4):1439-44, Pan et al (2011) Int J Cancer. 128(10):2251-60 and Saunders-Pullman et al (2010) Mov Disord. 2010 25(15):2536-41). In an analysis of Ashkanezi Jewish PD patients, those patients harboring the LRRK2 G2019S mutations were three times more likely to have non-skin cancers than those without this mutation, they were diagnosed with their cancers approximately 6 years earlier than other PD/cancer patients not carrying the G2019S mutation, and had a higher prevalence of developing their cancers prior to the PD than other PD/cancer patients (Saunders-Pullman ibid). In melanoma patients, there appears to be a correlation between PD and melanoma such that patients with PD have a higher prevalence of melanoma than the general population, and melanoma patients have a higher prevalence of PD (Pan et al, ibid). It has also been shown in some cancers, there is an amplification of the receptor tyrosine kinase MET which leads to increased receptor density and signaling even in the absence of the MET HGF (hepatocyte growth factor) ligand. Subsequent investigation found that a co-amplification of LRRK2 was present in these tumors, and, that repression of the LRRK2 amplification lead to a cessation of downstream mTOR and STAT3 signaling by MET. Thus, it appears that the HGF requirement for increased cell division signaling is somehow overcome by the LRRK2 co-amplification in some cancers (Loovenga et al, ibid).

Thus, there remains a need for the development of novel anti-PD and anti-cancer strategies, including treatments and model systems (in vitro and animal) to model and treat PD and/or cancer based on investigation of LRRK2 mutations.

### SUMMARY

Disclosed herein are methods and compositions for altering the PD related genetic loci LRRK2. Also described are models for studying the function of the PD-related gene LRRK2, models for PD drug and treatment discovery, as well as methods of making and using these model systems. Additionally described herein are models for studying the role of LRRK2 in cancer, and the development of models for cancer drug discovery relating to LRRK2. The compositions and methods described herein can be used for genome editing of LRRK2, including, but not limited to: cleaving of an LRRK2 in an animal cell resulting in targeted alteration (insertion, deletion and/or substitution mutations) in the LRRK2 gene, including the incorporation of these targeted alterations into the germline; targeted introduction into an LRRK2 gene of non-endogenous nucleic acid sequences, the partial or complete inactivation of an LRRK2 in an animal; methods of inducing homology-directed repair at an LRRK2 locus; and generation of transgenic animals (e.g., rodents and non-human primates).

In one aspect, described herein is a method of modifying, using a zinc finger nuclease (ZFN), a LRRK2 gene (e.g., an endogenous LRRK2 gene) in a cell or cell line. In certain embodiments, the cell or cell line is a fibroblast or iPSC and comprises the G2019S mutant LRRK2 protein encoded at the mutant LRRK2 gene. In certain embodiments, two ZFNs that bind to first and second target sites and form a dimer upon binding are used to cleave the LRRK2 gene between the first and second target sites. In any of the methods described herein, cleavage results in modification of the gene sequence by non-homologous end joining and/or by homology directed repair. Furthermore, any of the methods described herein may further comprise introducing into the cell an exogenous sequence wherein cleavage by the ZFN(s) results in for integration (insertion) of an exogenous sequence into the LRRK2 gene. The modifying may result in a cell that is heterozygous or homozygous for the modification, for example the modification may correct a G2019S encoded mutation in the cell and/or may introduce one or more additional mutations into the LRRK2 gene. In certain embodiments, the methods further comprise differentiating the ZFN-modified cells into dopaminergic neurons. Also provided are fibroblasts, iPSCs or dopaminergic neurons produced by the methods described herein (e.g., fibroblast, iPSC, neuronal cells or cell lines comprising at least two mutations in the LRRK2 gene).

In another aspect, described herein is a zinc-finger protein (ZFP) that binds to target site in a LRRK2 gene in a genome, wherein the ZFP comprises one or more engineered zinc-finger binding domains. In one embodiment, the ZFP is a zinc-finger nuclease (ZFN) that cleaves a target genomic region of interest, wherein the ZFN comprises one or more engineered zinc-finger binding domains and a nuclease cleavage domain or cleavage half-domain. Cleavage domains and cleavage half domains can be obtained, for example, from various restriction endonucleases and/or homing endonucleases. In one embodiment, the cleavage half-domains are derived from a Type IIS restriction endonuclease (e.g., Fok I). In certain embodiments, the zinc finger domain recognizes a target site in a LRRK2 gene.

The ZFN may bind to and/or cleave a LRRK2 gene within the coding region of the gene or in a non-coding sequence within or adjacent to the gene, such as, for example, a leader sequence, trailer sequence or intron, or within a non-transcribed region, either upstream or downstream of the coding region.

In another aspect, described herein is a TALE protein (Transcription activator like) that binds to target site in a LRRK2 gene in a genome, wherein the TALE comprises one or more engineered TALE binding domains. In one embodiment, the TALE is a nuclease (TALEN) that cleaves a target genomic region of interest, wherein the TALEN comprises one or more engineered TALE DNA binding domains and a nuclease cleavage domain or cleavage half-domain. Cleavage domains and cleavage half domains can be obtained, for example, from various restriction endonucleases and/or homing endonucleases. In one embodiment, the cleavage half-domains are derived from a Type IIS restriction endonuclease (e.g., Fok I). In certain embodiments, the TALE DNA binding domain recognizes a target site in a LRRK2 gene.

The TALEN may bind to and/or cleave a LRRK2 gene within the coding region of the gene or in a non-coding sequence within or adjacent to the gene, such as, for example, a leader sequence, trailer sequence or intron, or within a non-transcribed region, either upstream or downstream of the coding region.

In another aspect, described herein are compositions comprising one or more of the zinc-finger and/or TALE nucleases described herein. In certain embodiments, the composition comprises one or more zinc-finger or TALE nucleases in combination with a pharmaceutically acceptable excipient.

In another aspect, described herein is a polynucleotide encoding one or more ZFNs or TALENs described herein. The polynucleotide may be, for example, mRNA.

In another aspect, described herein is a ZFN or TALEN expression vector comprising a polynucleotide, encoding one or more ZFNs or TALENs described herein, operably linked to a promoter.

In another aspect, described herein is a host cell comprising one or more ZFN or TALEN expression vectors. The host cell may be stably transformed or transiently transfected or a combination thereof with one or more ZFP or TALEN expression vectors. In one embodiment, the host cell is an embryonic stem cell. In other embodiments, the one or more ZFP or TALEN expression vectors express one or more ZFNs or TALENs in the host cell. In another embodiment, the host cell may further comprise an exogenous polynucleotide donor sequence. In any of the embodiments, described herein, the host cell can comprise an embryo cell, for example a one or more mouse, rat, rabbit or other mammal cell embryo (e.g., a non-human primate).

In another aspect, described herein is a method for cleaving one or more LRRK2 genes in a cell, the method comprising: (a) introducing, into the cell, one or more polynucleotides encoding one or more ZFNs or TALENs that bind to a target site in the one or more genes under conditions such that the ZFN(s) is (are) or TALENs is (are) expressed and the one or more genes are cleaved.

In another embodiment, described herein is a method for modifying one or more LRRK2 gene sequence(s) in the genome of cell, the method comprising (a) providing a cell comprising one or more LRRK2 sequences; and (b) expressing first and second zinc-finger nucleases (ZFNs) or TALENs in the cell, wherein the first ZFN or TALEN binds to (and/or cleaves) at a first site and the second ZFN or TALEN binds to (and/or cleaves) at a second site, wherein the gene sequence is located between the first and second sites, wherein cleavage at the first and/or second sites results in modification of the gene sequence by non-homologous end joining and/or homology directed repair. Optionally, the cleavage results in insertion of an exogenous sequence (transgene) also introduced into the cell. In other embodiments, non-homologous end joining results in a deletion between the first and second sites. The size of the deletion in the gene sequence is determined by the distance between the first and second cleavage sites. Accordingly, deletions of any size, in any genomic region of interest, can be obtained. Deletions of 1, 5, 10, 25, 50, 100, 200, 300, 400, 500, 600, 700, 800, 900, 1,000 nucleotide pairs, or any integral value of nucleotide pairs within this range, can be obtained. In addition deletions of a sequence of any integral value of nucleotide pairs greater than 1,000 nucleotide pairs can be obtained using the methods and compositions disclosed herein. Using these methods and compositions, mutant LRRK2 proteins may be developed that lack one or more of the known domains. These constructs can then be used to study the function of the protein within a cell.

In another aspect, specific mutations associated with LRRK2 can be corrected to understand the function of the gene that harbors the mutation, and/or to discover phenotypes associated with the correction of the mutant gene. Such an understanding then can be used to design cells, cell lines and transgenic animals for use in drug screening and drug discovery, for example for treatments of PD and/or cancer.

In another aspect, site specific mutations in LRRK2 can be constructed to model known or novel mutations. For example, the G2019S mutation in LRRK2 can be constructed in a cell, cell line, primary cell or transgenic animal. In one embodiment, a cell, cell line or transgenic animal carrying a heterozygous genotype for LRRK2 is constructed, while in another embodiment, a homozygous cell, cell line or transgenic animal is made carrying two mutant copies in both alleles of a desired locus.

In another aspect, described herein are methods of inactivating a LRRK2 gene in a cell by introducing one or more proteins, polynucleotides and/or vectors into the cell as described herein. In any of the methods described herein the ZFNs and/or TALENs may induce targeted mutagenesis, targeted deletions of cellular DNA sequences, and/or facilitate targeted recombination at a predetermined chromosomal locus. Thus, in certain embodiments, the ZFNs and/or TALENs delete or insert one or more nucleotides of the target gene. In some embodiments, the LRRK2 gene is inactivated by ZFN or TALEN cleavage followed by non-homologous end joining (NHEJ). In other embodiments, a genomic sequence in the target gene is replaced, for example using a ZFN or TALEN (or vector encoding said ZFN or TALEN) as described herein and a “donor” sequence that is inserted into the gene following targeted cleavage with the ZFN or TALEN. The donor sequence may be present in the ZFN or TALEN vector, present in a separate vector (e.g., Ad or LV vector) or, alternatively, may be introduced into the cell using a different nucleic acid delivery mechanism. In one aspect, the donor sequence causes a known mutation, e.g., the G2019S mutation in the LRRK2 protein. In other aspects, other well known LRRK2 mutations can be constructed through gene modification (e.g. G2385R, R1441C, R1628P and others known in the art).

In another aspect, described herein are methods of correcting a LRRK2 gene in a cell by introducing one or more proteins, polynucleotides and/or vectors into the cell as described herein. In any of the methods described herein the ZFNs or TALENs may induce targeted mutagenesis, targeted deletions of cellular DNA sequences, and/or facilitate targeted recombination at a predetermined chromosomal locus. Thus, in certain embodiments, the ZFNs or TALENs delete or insert one or more nucleotides of the target gene. In some embodiments the LRRK2 gene is corrected by ZFN or TALEN cleavage followed by non-homologous end joining (NHEJ). In other embodiments, a genomic sequence in the target gene is replaced, for example using a ZFN or TALEN (or vector encoding said ZFN or TALEN) as described herein and a “donor” sequence that is integrated into the gene following targeted cleavage with the ZFN or TALEN correcting the sequence of the LRRK2 gene. Accordingly, also provided by the invention is a donor molecule which can comprise a transgene or portions thereof, regions of homology with the genome flanking the cleavage site, or various nucleic acid sequences associated with gene regulation (promoters and the like). The donor sequence may be present in the ZFN or TALEN vector, present in a separate vector (e.g., Ad or LV vector) or, alternatively, may be introduced into the cell using a different nucleic acid delivery mechanism. In one aspect, the donor sequence corrects a known mutation, e.g., ‘A’ guanine to ‘G’ at position 6055 in a mutant LRRK2 gene to cause the correction of the G2019S mutation. In other aspects, other well known LRRK2 mutation proteins can be corrected through gene modification (e.g. G2385R, R1441C, R1628P and others known in the art).

In some aspects, the cell is a stem cell. Specific stem cell types that may used with the methods and compositions of the invention include embryonic stem cells (ESC), hematopoietic stem cells, nerve stem cells, skin stem cells, muscle stem cells and induced pluripotent stem cells (iPSCs). The iPSCs can be derived from patient samples (e.g., fibroblasts) and from normal controls wherein the patient derived iPSC can be mutated to normal gene sequence at the gene of interest, or normal cells can be altered to the known disease allele at the gene of interest. Panels of these iPSC can be used to create isogenic cells with both patient and normal cells carrying one or more mutations at their endogenous LRRK2 loci. These cells can be used to create cell lines and/or transgenic animals bearing several mutations of interest to study multigene affects of disease severity and possible therapeutic treatments, including for example for PD and/or cancer. Other cell types that may be used for these studies are patient derived fibroblasts, or other types of patient cells such as neurons.

In yet another aspect, described herein is a method for germline disruption of one or more target LRRK2 genes, the method comprising modifying one or more LRRK2 sequences in the genome of one or more cells of an embryo by any of the methods described herein and allowing the embryo to develop, wherein that the modified gene sequences are present in at least a portion of gametes of the sexually mature animal. In certain embodiments, the animal is a small mammal, such as a rodent or rabbit. In some embodiments, the animal is a non-human primate.

In another aspect, described herein is a method of creating one or more heritable mutant alleles in at least one LRRK2 locus of interest, the method comprising modifying one or more LRRK2 loci in the genome of one or more cells of an animal embryo by any of the methods described herein; raising the embryo to sexual maturity; and allowing the sexually mature animal to produce offspring; wherein at least some of the offspring comprise the mutant alleles. In certain embodiments, the animal is a small mammal, for example a rabbit or a rodent such as rat, a mouse or a guinea pig. In other embodiments, the animal is a non-human primate.

In any of the methods described herein, the polynucleotide encoding the zinc finger nuclease(s) or TALEN(s) can comprise DNA, RNA or combinations thereof. In certain embodiments, the polynucleotide comprises a plasmid. In other embodiments, the polynucleotide encoding the nuclease comprises mRNA.

In a still further aspect, provided herein is a method for site specific integration of a nucleic acid sequence into a LRRK2 locus of a chromosome. In certain embodiments, the method comprises: (a) injecting an embryo with (i) at least one DNA vector, wherein the DNA vector comprises an upstream sequence and a downstream sequence flanking the donor nucleic acid sequence to be integrated, and (ii) at least one RNA molecule encoding a zinc finger or TALE nuclease that recognizes the site of integration in the LRRK2 locus, and (b) culturing the embryo to allow expression of the zinc finger or TALE nuclease, wherein a double stranded break introduced into the site of integration by the zinc finger nuclease or TALEN is repaired, via homologous recombination with the DNA vector, so as to integrate the nucleic acid sequence into the chromosome.

Suitable embryos may be derived from several different vertebrate species, including mammalian, bird, reptile, amphibian, and fish species. Generally speaking, a suitable embryo is an embryo that may be collected, injected, and cultured to allow the expression of a zinc finger or TALE nuclease. In some embodiments, suitable embryos may include embryos from small mammals (e.g., rodents, rabbits, etc.), companion animals, livestock, and primates. Non-limiting examples of rodents may include mice, rats, hamsters, gerbils, and guinea pigs. Non-limiting examples of companion animals may include cats, dogs, rabbits, hedgehogs, and ferrets. Non-limiting examples of livestock may include horses, goats, sheep, swine, llamas, alpacas, and cattle. Non-limiting examples of primates may include capuchin monkeys, chimpanzees, lemurs, macaques, marmosets, tamarins, spider monkeys, squirrel monkeys, and vervet monkeys. In other embodiments, suitable embryos may include embryos from fish, reptiles, amphibians, or birds. Alternatively, suitable embryos may be insect embryos, for instance, a Drosophila embryo or a mosquito embryo.

Also provided is an embryo comprising at least one DNA vector, wherein the DNA vector comprises an upstream sequence and a downstream sequence flanking the nucleic acid sequence to be integrated, and at least one RNA molecule encoding a zinc finger nuclease that recognizes the chromosomal site of integration. Organisms derived from any of the embryos as described herein are also provided.

In another aspect provided by the methods and compositions of the invention is the use of cells, cell lines and animals (e.g., transgenic animals) in the screening of drug libraries and/or other therapeutic compositions (i.e., antibodies, structural RNAs, etc.) for use in treatment of an animal afflicted with PD and/or cancer. Such screens can begin at the cellular level with manipulated cell lines or primary cells, and can progress up to the level of treatment of a whole animal.

A kit, comprising the ZFPs or TALENs of the invention, is also provided. The kit may comprise nucleic acids encoding the ZFPs or TALENs, (e.g. RNA molecules or ZFP or TALEN encoding genes contained in a suitable expression vector), donor molecules, suitable host cell lines, instructions for performing the methods of the invention, and the like.

These and other aspects will be readily apparent to the skilled artisan in light of disclosure as a whole.

### BRIEF DESCRIPTION OF THE DRAWINGS

FIG. 1 depicts a gel showing the results of a Cel-I mismatch assay on K562 cells treated with ZFNs targeting the LRRK2 gene. All samples were ZFN pairs where one member of the pair is always the ZFN SBS 27870. The lanes show the results where the other member of the ZFN pair was varied. The Cel-I assay (Surveyor™, Transgenomic) identifies mismatches in a DNA sequence that occur as a result of cleavage followed by repair of the DSB by the NHEJ cellular machinery. In the NHEJ process, small insertions and deletions are often observed (indels), and these create the mismatches identified in the Cel-I assay. At the bottom of each lane is shown the percent of the sequences that display some amount of NHEJ-caused indels at the targeted location. The arrow indicates the band observed from the cleavage by the Surveyor™ enzyme of the mismatch bubble.

FIG. 2 shows a gel depicting results of an experiment designed to knock in the 6055G>A mutation to create the G2019S allele in the LRRK2 gene. In this experiment, ZFNs and donor plasmid containing the sequence for the mutated allele were nucleofected into K562 cells. The DNA was isolated, and a PCR reaction was performed to amplify the region around the mutant locus. The product was then digested with SfcI, and run on a gel. The presence of the mutation creates an SfcI site therefore results in a faster migrating band as described above. The faster migrating band is indicated with an arrow while the wild type band is indicated with an open triangle.

FIG. 3, panels A and B, depict gels for genotyping ZFN-modified fibroblast single cell clones. Patient fibroblast pools were treated with LRRK2-specific ZFNs and donor DNA which could cause a gene correction event if incorporated. After treatment, single clone colonies were isolated from the pools and analyzed by PCR and enzymatic digestion for evidence of ZFN induced modification. FIG. 3A depicts the results of 36 single cell clones analyzed by PCR and BsrDI digestion. Digestion of the PCR product with this enzyme will result in two known bands (the BsrD1 site is between the 2 ZFN binding sites), and any deviation from this pattern could indicate the presence of an NHEJ event. FIG. 3B depicts the results of the analysis using the SfcI enzyme, which is created by the G2019S encoded mutation. In cells that are heterozygous for the mutation, the wild-type allele is resistant to SfcI digest (top band), and the mutant allele can be cleaved by SfcI, which yields the two faster migrating bands, the loss of which can indicate the presence of a gene correction event. The data presented in FIG. 3 is consistent with nuclease induced NHEJ occurring, but gene correction was not detected in this small number of clones.

FIG. 4 depicts a gel used for genotyping ZFN-modified iPSCs. Fibroblasts were treated with ZFNs and donor as described previously, and then induced to become iPSC using standard protocols (Takahashi and Yamanaka (2006) Cell 126(4): 663-676). DNA from the iPSC clones was isolated, and subjected to PCR amplification. Digestion of the PCR products was done as before for FIG. 3, and the results are shown. It is apparent that ZFN-induced NHEJ has occurred.

FIG. 5, panels A and B, show ZFN modification of LRRK2 in patient derived fibroblasts and iPSCs. FIG. 5A depicts a gel showing the results of a Cel-I mismatch assay on patient derived fibroblasts treated with ZFNs targeting the LRRK2 gene. The Cel-I assay (Surveyor™, Transgenomic) identifies mismatches in a DNA sequence that occur as a result of cleavage followed by repair of the DSB by the NHEJ cellular machinery. In the NHEJ process, small insertions and deletions are often observed (indels), and these create the mismatches identified in the Cel-I assay. “ZFN” indicates ZFN-treated samples and “GFP” indicates control GFP samples. The top band represents uncut PCR products, the cleavage products are indicate by arrows. For patient-derived fibroblasts, “*” denotes cleavage products resulted from the heteroduplex between wild type and unmodified G2019S alleles. The relative intensity of the cleavage products compared with the parental band was quantitated by densitometryis, and provides a measure of the frequency of ZFN-mediated cleavage of the LRRK2 gene, approximately 16% in fibroblasts. FIG. 5B depicts a gel showing the results of a Cel-I mismatch assay on patient derived iPSC treated with ZFNs targeting the LRRK2 gene. As for panel 5A, “*” denotes the cleavage products resulting from the heteroduplex that formed between the wild type and the unmodified G20219S mutant alleles, while the arrows indicate the cleavage products that occur due to the NHEJ at the cleavage site.

FIG. 6 depicts a gel displaying the results of a Cel-I mismatch assay on patient derived iPSCs treated with ZFN pairs and donor. Above the gel, a grid of the components of each reaction is shown, which indicates which ZFN pair was used, and whether donor DNA was included. In addition, the gel shows the results found on cultures derived from single wells, or pools of transfected cells. The percent gene modification (“% Indels) is shown at the bottom of the gel. The arrows indicate the bands produced due to ZFN induced gene modification, while the asterisks (“*”) indicate as above the bands resulting from the heteroduplex that formed between the wild type and the G20219S mutant alleles.

FIG. 7, panels A to C, depict gels showing the results of restriction digests of PCR products that contain the G2019S mutation and the corresponding wild-type sequence following ZFN/donor treatment. FIG. 7A depicts the results of digestion with BsrDI. This enzyme cleaves in between the two ZFP binding sites, and so loss of the wild type band (indicated at the right) demonstrates that NHEJ or HDR has occurred. FIG. 7B depicts the results following restriction with AciI. The AciI restriction site is introduced into the donor construct as a silent mutation (no codon change) that allows the detection of integrated donor; the presence of AciI site suggests cells have undergone HDR-mediated donor insertion (gene correction). Clones 7, 11 and 18 have a new band indicating that the AciI restriction site has been inserted via by HDR. Gene correction of G2019S mutation results in the loss of the SfcI site in the mutant sequence, and FIG. 7C depicts the results from a SfcI restriction digest. Clones 7 and 11 lack the mutant SfcI site and thus have undergone gene correction.

### DETAILED DESCRIPTION

Disclosed herein are compositions and methods for treating and/or developing models (in vitro and in vivo) useful in evaluating treatment of PD. In particular, nuclease-mediated cleavage and integration is used to create or repair known mutations in the LRRK2 gene. These compositions and methods can be used to correct and/or create specific LRRK2 mutations in any selected genetic background to allow for the treatment and/or study of multifactorial interaction in PD. The compositions and methods described herein can be used to create isogenic panels of a set of mutations in LRRK2 to allow for controlled study of these mutations, to investigate the link between a certain mutation and cellular dysfunction and to identify phenotypes associated with the mutation or with the correction of the mutation. In addition, any LRRK2 mutation can be introduced into patient derived cells, e.g. patient derived induced pluripotent stem cells, fibroblasts or neurons to investigate the affects of a certain mutation in a patient cell background. In addition, creation of LRRK2 mutants with in-frame alterations is also part of the invention described herein, to allow for fine tuned analysis of the functional domains of this protein. In addition, LRRK2 mutations associated with PD can be created within the native gene in model animals (rat, non-human primate, etc.) to generate PD models. These animals may contain one or more inserted LRRK2 mutations.

Also described herein are methods and compositions for altering specific LRRK2 defects in patient cells. For example, mutated LRRK2 genes may be knocked out by use of specific nucleases that will only act on mutant alleles and not act on a wild type gene sequence. Knock out of a specific gene may be a result of cleavage followed by NHEJ, or by cleavage at two loci within the gene to delete a large portion of the gene, or by cleavage followed by targeted integration of an oligonucleotide or larger donor DNA.

General

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, for example, Sambrook et al. MOLECULAR CLONING: A LABORATORY MANUAL, Second edition, Cold Spring Harbor Laboratory Press, 1989 and Third edition, 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolffe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P.B. Becker, ed.) Humana Press, Totowa, 1999.

### DEFINITIONS

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of corresponding naturally-occurring amino acids.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

Zinc finger binding domains or TALEN can be “engineered” to bind to a predetermined nucleotide sequence, for example via engineering (altering one or more amino acids) of the recognition helix region of a naturally occurring zinc finger or by engineering the RVDs of a TALEN protein. Therefore, engineered zinc finger proteins or TALENs are proteins that are non-naturally occurring. Non-limiting examples of methods for engineering zinc finger or TALEN proteins are design and selection. A designed zinc finger or TALEN protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536; WO 03/016496 and WO 2011/146121.

A “selected” zinc finger or TALEN protein is a protein not found in nature whose production results primarily from an empirical process such as phage display, interaction trap or hybrid selection. See e.g., U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970; WO 01/88197; WO 02/099084 and WO 2011/146121.

“Recombination” refers to a process of exchange of genetic information between two polynucleotides. For the purposes of this disclosure, “homologous recombination (BR)” refers to the specialized form of such exchange that takes place, for example, during repair of double-strand breaks in cells via homology-directed repair mechanisms. This process requires nucleotide sequence homology, uses a “donor” molecule to template repair of a “target” molecule (i.e., the one that experienced the double-strand break), and is variously known as “non-crossover gene conversion” or “short tract gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to re-synthesize genetic information that will become part of the target, and/or related processes. Such specialized HR often results in an alteration of the sequence of the target molecule such that part or all of the sequence of the donor polynucleotide is incorporated into the target polynucleotide.

In the methods of the disclosure, one or more targeted nucleases as described herein create a double-stranded break in the target sequence (e.g., cellular chromatin) at a predetermined site, and a “donor” polynucleotide, having homology to the nucleotide sequence in the region of the break, can be introduced into the cell. The presence of the double-stranded break has been shown to facilitate integration of the donor sequence. The donor sequence may be physically integrated or, alternatively, the donor polynucleotide is used as a template for repair of the break via homologous recombination, resulting in the introduction of all or part of the nucleotide sequence as in the donor into the cellular chromatin. Thus, a first sequence in cellular chromatin can be altered and, in certain embodiments, can be converted into a sequence present in a donor polynucleotide. Thus, the use of the terms “replace” or “replacement” can be understood to represent replacement of one nucleotide sequence by another, (i.e., replacement of a sequence in the informational sense), and does not necessarily require physical or chemical replacement of one polynucleotide by another.

In any of the methods described herein, additional pairs of zinc-finger or TALEN proteins can be used for additional double-stranded cleavage of additional target sites within the cell.

In certain embodiments of methods for targeted recombination and/or replacement and/or alteration of a sequence in a region of interest in cellular chromatin, a chromosomal sequence is altered by homologous recombination with an exogenous “donor” nucleotide sequence. Such homologous recombination is stimulated by the presence of a double-stranded break in cellular chromatin, if sequences homologous to the region of the break are present.

In any of the methods described herein, the first nucleotide sequence (the “donor sequence”) can contain sequences that are homologous, but not identical, to genomic sequences in the region of interest, thereby stimulating homologous recombination to insert a non-identical sequence in the region of interest. Thus, in certain embodiments, portions of the donor sequence that are homologous to sequences in the region of interest exhibit between about 80 to 99% (or any integer therebetween) sequence identity to the genomic sequence that is replaced. In other embodiments, the homology between the donor and genomic sequence is higher than 99%, for example if only 1 nucleotide differs as between donor and genomic sequences of over 100 contiguous base pairs. In certain cases, a non-homologous portion of the donor sequence can contain sequences not present in the region of interest, such that new sequences are introduced into the region of interest. In these instances, the non-homologous sequence is generally flanked by sequences of 50-1,000 base pairs (or any integral value therebetween) or any number of base pairs greater than 1,000, that are homologous or identical to sequences in the region of interest. In other embodiments, the donor sequence is non-homologous to the first sequence, and is inserted into the genome by non-homologous recombination mechanisms.

Any of the methods described herein can be used for partial or complete inactivation of one or more target sequences in a cell by targeted integration of donor sequence that disrupts expression of the gene(s) of interest. Cell lines with partially or completely inactivated genes are also provided.

Furthermore, the methods of targeted integration as described herein can also be used to integrate one or more exogenous sequences. The exogenous nucleic acid sequence can comprise, for example, one or more genes or cDNA molecules, or any type of coding or non-coding sequence, as well as one or more control elements (e.g., promoters). In addition, the exogenous nucleic acid sequence may produce one or more RNA molecules (e.g., small hairpin RNAs (shRNAs), inhibitory RNAs (RNAis), microRNAs (miRNAs), etc.).

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value therebetween or thereabove), preferably between about 100 and 1,000 nucleotides in length (or any integer therebetween), more preferably between about 200 and 500 nucleotides in length.

“Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic. Cellular chromatin includes both chromosomal and episomal chromatin.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes.

An “episome” is a replicating nucleic acid, nucleoprotein complex or other structure comprising a nucleic acid that is not part of the chromosomal karyotype of a cell. Examples of episomes include plasmids and certain viral genomes.

A “target site” or “target sequence” is a nucleic acid sequence that defines a portion of a nucleic acid to which a binding molecule will bind, provided sufficient conditions for binding exist.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present only during embryonic development of muscle is an exogenous molecule with respect to an adult muscle cell. Similarly, a molecule induced by heat shock is an exogenous molecule with respect to a non-heat-shocked cell. An exogenous molecule can comprise, for example, a functioning version of a malfunctioning endogenous molecule or a malfunctioning version of a normally-functioning endogenous molecule.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, lipid-mediated transfer (i.e., liposomes, including neutral and cationic lipids), electroporation, direct injection, cell fusion, particle bombardment, calcium phosphate co-precipitation, DEAE-dextran-mediated transfer and viral vector-mediated transfer. An exogenous molecule can also be the same type of molecule as an endogenous molecule but derived from a different species than the cell is derived from. For example, a human nucleic acid sequence may be introduced into a cell line originally derived from a mouse or hamster.

By contrast, an “endogenous” molecule is one that is normally present in a particular cell at a particular developmental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins (for example, a fusion between a ZFP or TALE DNA-binding domain and one or more activation domains) and fusion nucleic acids (for example, a nucleic acid encoding the fusion protein described supra). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of an mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression. Genome editing (e.g., cleavage, alteration, inactivation, random mutation) can be used to modulate expression. Gene inactivation refers to any reduction in gene expression as compared to a cell that does not include a ZFP or TALEN as described herein. Thus, gene inactivation may be partial or complete.

A “region of interest” is any region of cellular chromatin, such as, for example, a gene or a non-coding sequence within or adjacent to a gene, in which it is desirable to bind an exogenous molecule. Binding can be for the purposes of targeted DNA cleavage and/or targeted recombination. A region of interest can be present in a chromosome, an episome, an organellar genome (e.g., mitochondrial, chloroplast), or an infecting viral genome, for example. A region of interest can be within the coding region of a gene, within transcribed non-coding regions such as, for example, leader sequences, trailer sequences or introns, or within non-transcribed regions, either upstream or downstream of the coding region. A region of interest can be as small as a single nucleotide pair or up to 2,000 nucleotide pairs in length, or any integral value of nucleotide pairs.

“Eukaryotic” cells include, but are not limited to, fungal cells (such as yeast), plant cells, animal cells, mammalian cells and human cells (e.g., T-cells).

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a ZFP or TALE DNA-binding domain is fused to an activation domain, the ZFP or TALE DNA-binding domain and the activation domain are in operative linkage if, in the fusion polypeptide, the ZFP or TALE DNA-binding domain portion is able to bind its target site and/or its binding site, while the activation domain is able to up-regulate gene expression. When a fusion polypeptide in which a ZFP or TALE DNA-binding domain is fused to a cleavage domain, the ZFP or TALE DNA-binding domain and the cleavage domain are in operative linkage if, in the fusion polypeptide, the ZFP or TALE DNA-binding domain portion is able to bind its target site and/or its binding site, while the cleavage domain is able to cleave DNA in the vicinity of the target site.

A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning, and expression vehicles, as well as integrating vectors.

A “reporter gene” or “reporter sequence” refers to any sequence that produces a protein product that is easily measured, preferably although not necessarily in a routine assay. Suitable reporter genes include, but are not limited to, sequences encoding proteins that mediate antibiotic resistance (e.g., ampicillin resistance, neomycin resistance, G418 resistance, puromycin resistance), sequences encoding colored or fluorescent or luminescent proteins (e.g., green fluorescent protein, enhanced green fluorescent protein, red fluorescent protein, luciferase), and proteins which mediate enhanced cell growth and/or gene amplification (e.g., dihydrofolate reductase). Epitope tags include, for example, one or more copies of FLAG, His, myc, Tap, HA or any detectable amino acid sequence. “Expression tags” include sequences that encode reporters that may be operably linked to a desired gene sequence in order to monitor expression of the gene of interest.

Nucleases

Described herein are compositions, particularly nucleases, which are useful in correction of a mutant LRRK2 allele and/or mutation of an LRRK2 allele, for example to generate models of PD and/or cancer. In certain embodiments, the nuclease is naturally occurring. In other embodiments, the nuclease is non-naturally occurring, i.e., engineered in the DNA-binding domain and/or cleavage domain. For example, the DNA-binding domain of a naturally-occurring nuclease may be altered to bind to a selected target site (e.g., a meganuclease that has been engineered to bind to site different than the cognate binding site). In other embodiments, the nuclease comprises heterologous DNA-binding and cleavage domains (e.g., zinc finger nucleases (ZFNs); TAL-effector nucleases (TALENs); meganuclease DNA-binding domains with heterologous cleavage domains).

A. DNA-Binding Domains

In certain embodiments, the nuclease is a meganuclease (homing endonuclease). Naturally-occurring meganucleases recognize 15-40 base-pair cleavage sites and are commonly grouped into four families: the LAGLIDADG family, the GIY-YIG family, the His-Cyst box family and the HNH family. Exemplary homing endonucleases include I-SceI, I-CeuI, PI-PspI, PI-Sce, I-SceIV, I-CsmI, I-PanI, I-SceII, I-PpoI, I-SceIII, I-CreI, I-TevI, I-TevII and I-TevIII. Their recognition sequences are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue.

In certain embodiments, the nuclease comprises an engineered (non-naturally occurring) homing endonuclease (meganuclease). The recognition sequences of homing endonucleases and meganucleases such as I-SceI, I-CeuI, PI-PspI, PI-Sce, I-SceIV, I-SceIII, I-CreI, I-TevI, I-TevII and I-TevIII are known. See also U.S. Pat. No. 5,420,032; U.S. Pat. No. 6,833,252; Belfort et al. (1997) Nucleic Acids Res. 25:3379-3388; Dujon et al. (1989) Gene 82:115-118; Perler et al. (1994) Nucleic Acids Res. 22, 1125-1127; Jasin (1996) Trends Genet. 12:224-228; Gimble et al. (1996) J. Mol. Biol. 263:163-180; Argast et al. (1998) J. Mol. Biol. 280:345-353 and the New England Biolabs catalogue. In addition, the DNA-binding specificity of homing endonucleases and meganucleases can be engineered to bind non-natural target sites. See, for example, Chevalier et al. (2002) Molec. Cell 10:895-905; Epinat et al. (2003) Nucleic Acids Res. 31:2952-2962; Ashworth et al. (2006) Nature 441:656-659; Paques et al. (2007) Current Gene Therapy 7:49-66; U.S. Patent Publication No. 20070117128. The DNA-binding domains of the homing endonucleases and meganucleases may be altered in the context of the nuclease as a whole (i.e., such that the nuclease includes the cognate cleavage domain) or may be fused to a heterologous cleavage domain.

In other embodiments, the DNA-binding domain comprises a naturally occurring or engineered (non-naturally occurring) TAL effector DNA binding domain. See, e.g., U.S. Patent Publication No. 20110301073, incorporated by reference in its entirety herein. The plant pathogenic bacteria of the genus Xanthomonas are known to cause many diseases in important crop plants. Pathogenicity of Xanthomonas depends on a conserved type III secretion (T3S) system which injects more than 25 different effector proteins into the plant cell. Among these injected proteins are transcription activator-like effectors (TALE) which mimic plant transcriptional activators and manipulate the plant transcriptome (see Kay et al (2007) Science 318:648-651). These proteins contain a DNA binding domain and a transcriptional activation domain. One of the most well characterized TALEs is AvrBs3 from Xanthomonas campestgris pv. Vesicatoria (see Bonas et al (1989) Mol Gen Genet 218: 127-136 and WO2010079430). TALEs contain a centralized domain of tandem repeats, each repeat containing approximately 34 amino acids, which are key to the DNA binding specificity of these proteins. In addition, they contain a nuclear localization sequence and an acidic transcriptional activation domain (for a review see Schornack S, et al (2006) J Plant Physiol 163(3): 256-272). In addition, in the phytopathogenic bacteria Ralstonia solanacearum two genes, designated brg11 and hpx17 have been found that are homologous to the AvrBs3 family of Xanthomonas in the R. solanacearum biovar 1 strain GMI1000 and in the biovar 4 strain RS1000 (See Heuer et al (2007) Appl and Envir Micro 73(13): 4379-4384). These genes are 98.9% identical in nucleotide sequence to each other but differ by a deletion of 1,575 bp in the repeat domain of hpx17. However, both gene products have less than 40% sequence identity with AvrBs3 family proteins of Xanthomonas.

Specificity of these TALEs depends on the sequences found in the tandem repeats. The repeated sequence comprises approximately 102 bp and the repeats are typically 91-100% homologous with each other (Bonas et al, ibid). Polymorphism of the repeats is usually located at positions 12 and 13 and there appears to be a one-to-one correspondence between the identity of the hypervariable diresidues at positions 12 and 13 with the identity of the contiguous nucleotides in the TALE's target sequence (see Moscou and Bogdanove, (2009) Science 326:1501 and Boch et al (2009) Science 326:1509-1512). Experimentally, the code for DNA recognition of these TALEs has been determined such that an HD sequence at positions 12 and 13 leads to a binding to cytosine (C), NG binds to T, NI to A, C, G or T, NN binds to A or G, and IG binds to T. These DNA binding repeats have been assembled into proteins with new combinations and numbers of repeats, to make artificial transcription factors that are able to interact with new sequences and activate the expression of a non-endogenous reporter gene in plant cells (Boch et al, ibid). Engineered TAL proteins have been linked to a FokI cleavage half domain to yield a TAL effector domain nuclease fusion (TALEN) exhibiting activity in a yeast reporter assay (plasmid based target). Christian et al ((2010)<Genetics epub 10.1534/genetics.110.120717). See, also, U.S. Patent Publication No. 20110301073, incorporated by reference in its entirety.

In certain embodiments, the DNA binding domain comprises a zinc finger protein. Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, DNA domains (e.g., multi-fingered zinc finger proteins) may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The zinc finger proteins described herein may include any combination of suitable linkers between the individual zinc fingers of the protein. In addition, enhancement of binding specificity for zinc finger binding domains has been described, for example, in co-owned WO 02/077227.

Selection of target sites; ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970; WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

B. Cleavage Domains

Any suitable cleavage domain can be operatively linked to a DNA-binding domain to form a nuclease. For example, ZFP DNA-binding domains have been fused to nuclease domains to create ZFNs—a functional entity that is able to recognize its intended nucleic acid target through its engineered (ZFP) DNA binding domain and cause the DNA to be cut near the ZFP binding site via the nuclease activity. See, e.g., Kim et al. (1996) Proc Nat'l Acad Sci USA 93(3):1156-1160. More recently, ZFNs have been used for genome modification in a variety of organisms. See, for example, United States Patent Publications 20030232410; 20050208489; 20050026157; 20050064474; 20060188987; 20060063231; and International Publication WO 07/014,275.

As noted above, the cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., S1 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides or nucleotide pairs can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the FokI enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used. Exemplary Type IIS restriction enzymes are described in International Publication WO 07/014,275, incorporated herein in its entirety. Additional restriction enzymes also contain separable binding and cleavage domains, and these are contemplated by the present disclosure. See, for example, Roberts et al. (2003) Nucleic Acids Res. 31:418-420.

A cleavage domain or cleavage half-domain can be any portion of a protein that retains cleavage activity, or that retains the ability to multimerize (e.g., dimerize) to form a functional cleavage domain.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987; 20080131962 and 20110201055, the disclosures of all of which are incorporated by reference in their entireties herein. Amino acid residues at positions 446, 447, 479, 483, 484, 486, 487, 490, 491, 496, 498, 499, 500, 531, 534, 537, and 538 of Fok I are all targets for influencing dimerization of the Fok I cleavage half-domains.

Exemplary engineered cleavage half-domains of Fok I that form obligate heterodimers include a pair in which a first cleavage half-domain includes mutations at amino acid residues at positions 490 and 538 of Fok I and a second cleavage half-domain includes mutations at amino acid residues 486 and 499.

Thus, in one embodiment, a mutation at 490 replaces Glu (E) with Lys (K); the mutation at 538 replaces Iso (I) with Lys (K); the mutation at 486 replaced Gln (Q) with Glu (E); and the mutation at position 499 replaces Iso (I) with Lys (K). Specifically, the engineered cleavage half-domains described herein were prepared by mutating positions 490 (E→K) and 538 (I→K) in one cleavage half-domain to produce an engineered cleavage half-domain designated “E490K:I538K” and by mutating positions 486 (Q→E) and 499 (I→L) in another cleavage half-domain to produce an engineered cleavage half-domain designated “Q486E:I499L”. The engineered cleavage half-domains described herein are obligate heterodimer mutants in which aberrant cleavage is minimized or abolished. See, e.g., U.S. Patent Publication Nos. 2008/0131962, the disclosure of which is incorporated by reference in its entirety for all purposes. In certain embodiments, the engineered cleavage half-domain comprises mutations at positions 486, 499 and 496 (numbered relative to wild-type FokI), for instance mutations that replace the wild type Gln (Q) residue at position 486 with a Glu (E) residue, the wild type Iso (I) residue at position 499 with a Leu (L) residue and the wild-type Asn (N) residue at position 496 with an Asp (D) or Glu (E) residue (also referred to as a “ELD” and “ELE” domains, respectively). In other embodiments, the engineered cleavage half-domain comprises mutations at positions 490, 538 and 537 (numbered relative to wild-type FokI), for instance mutations that replace the wild type Glu (E) residue at position 490 with a Lys (K) residue, the wild type Iso (I) residue at position 538 with a Lys (K) residue, and the wild-type His (H) residue at position 537 with a Lys (K) residue or a Arg (R) residue (also referred to as “KKK” and “KKR” domains, respectively). In other embodiments, the engineered cleavage half-domain comprises mutations at positions 490 and 537 (numbered relative to wild-type FokI), for instance mutations that replace the wild type Glu (E) residue at position 490 with a Lys (K) residue and the wild-type His (H) residue at position 537 with a Lys (K) residue or a Arg (R) residue (also referred to as “KIK” and “KIR” domains, respectively). (See US Publication No. 2011/0201055).

Engineered cleavage half-domains described herein can be prepared using any suitable method, for example, by site-directed mutagenesis of wild-type cleavage half-domains (Fok I) as described in U.S. Patent Publication Nos. 20050064474 and 20080131962.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

Nucleases can be screened for activity prior to use, for example in a yeast-based chromosomal system as described in WO 2009/042163 and 20090068164. Nuclease expression constructs can be readily designed using methods known in the art. See, e.g., United States Patent Publications 20030232410; 20050208489; 20050026157; 20050064474; 20060188987; 20060063231; and International Publication WO 07/014,275. Expression of the nuclease may be under the control of a constitutive promoter or an inducible promoter, for example the galactokinase promoter which is activated (de-repressed) in the presence of raffinose and/or galactose and repressed in presence of glucose.

### Target Sites

As described in detail above, DNA domains can be engineered to bind to any sequence of choice in an LRRK2 locus. An engineered DNA-binding domain can have a novel binding specificity, compared to a naturally-occurring DNA-binding domain. Engineering methods include, but are not limited to, rational design and various types of selection. Rational design includes, for example, using databases comprising triplet (or quadruplet) nucleotide sequences and individual (e.g., zinc finger) amino acid sequences, in which each triplet or quadruplet nucleotide sequence is associated with one or more amino acid sequences of DNA binding domain which bind the particular triplet or quadruplet sequence. See, for example, co-owned U.S. Pat. Nos. 6,453,242 and 6,534,261, incorporated by reference herein in their entireties. Rational design of TAL-effector domains can also be performed. See, e.g., U.S. Patent Publication No. 20110301073.

Exemplary selection methods applicable to DNA-binding domains, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237.

Selection of target sites; nucleases and methods for design and construction of fusion proteins (and polynucleotides encoding same) are known to those of skill in the art and described in detail in U.S. Patent Application Publication Nos. 20050064474 and 20060188987, incorporated by reference in their entireties herein.

In addition, as disclosed in these and other references, DNA-binding domains (e.g., multi-fingered zinc finger proteins) may be linked together using any suitable linker sequences, including for example, linkers of 5 or more amino acids. See, e.g., U.S. Pat. Nos. 6,479,626; 6,903,185; and 7,153,949 for exemplary linker sequences 6 or more amino acids in length. The proteins described herein may include any combination of suitable linkers between the individual DNA-binding domains of the protein. See, also, U.S. Patent Publication No. 20110287512.

### Donors

As noted above, alteration of an LRRK2 gene can include insertion of an exogenous sequence (also called a “donor sequence” or “donor”). It will be readily apparent that the donor sequence is typically not identical to the genomic sequence that it replaces. For example, the sequence of the donor polynucleotide can contain one or more single base changes, insertions, deletions, inversions or rearrangements with respect to the genomic sequence, so long as sufficient homology with chromosomal sequences is present. Alternatively, a donor sequence can contain a non-homologous sequence flanked by two regions of homology. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest.

The donor polynucleotide can be DNA or RNA, single-stranded or double-stranded and can be introduced into a cell in linear or circular form. If introduced in linear form, the ends of the donor sequence can be protected (e.g., from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally inserted so that its expression is driven by the endogenous promoter at the integration site, namely the promoter that drives expression of the LRRK2 gene. However, it will be apparent that the donor may comprise a promoter and/or enhancer, for example a constitutive promoter or an inducible or tissue specific promoter.

Furthermore, although not required for expression, exogenous sequences may also be transcriptional or translational regulatory sequences, for example, promoters, enhancers, insulators, internal ribosome entry sites, sequences encoding 2A peptides and/or polyadenylation signals.

### Delivery

The nucleases, polynucleotides encoding these nucleases, donor polynucleotides and compositions comprising the proteins and/or polynucleotides described herein may be delivered in vivo or ex vivo by any suitable means.

Methods of delivering nucleases as described herein are described, for example, in U.S. Pat. Nos. 6,453,242; 6,503,717; 6,534,261; 6,599,692; 6,607,882; 6,689,558; 6,824,978; 6,933,113; 6,979,539; 7,013,219; and 7,163,824, the disclosures of all of which are incorporated by reference herein in their entireties.

Nucleases and/or donor constructs as described herein may also be delivered using vectors containing sequences encoding one or more of the zinc finger or TALEN protein(s). Any vector systems may be used including, but not limited to, plasmid vectors, retroviral vectors, lentiviral vectors, adenovirus vectors, poxvirus vectors; herpesvirus vectors and adeno-associated virus vectors, etc. See, also, U.S. Pat. Nos. 6,534,261; 6,607,882; 6,824,978; 6,933,113; 6,979,539; 7,013,219; and 7,163,824, incorporated by reference herein in their entireties. Furthermore, it will be apparent that any of these vectors may comprise one or more of the sequences needed for treatment. Thus, when one or more nucleases and a donor construct are introduced into the cell, the nucleases and/or donor polynucleotide may be carried on the same vector or on different vectors. When multiple vectors are used, each vector may comprise a sequence encoding one or multiple nucleases and/or donor constructs.

Conventional viral and non-viral based gene transfer methods can be used to introduce nucleic acids encoding nucleases and donor constructs in cells (e.g., mammalian cells) and target tissues. Non-viral vector delivery systems include DNA plasmids, naked nucleic acid, and nucleic acid complexed with a delivery vehicle such as a liposome or poloxamer. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Milani & Caskey, TIBTECH 11:162-166 (1993); Dillon, TIBTECH 11:167-175 (1993); Miller, Nature 357:455-460 (1992); Van Brunt, Biotechnology 6(10):1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology Doerfler and Böhm (eds.) (1995); and Yu et al., Gene Therapy 1:13-26 (1994).

Methods of non-viral delivery of nucleic acids include electroporation, lipofection, microinjection, biolistics, virosomes, liposomes, immunoliposomes, polycation or lipid:nucleic acid conjugates, naked DNA, artificial virions, and agent-enhanced uptake of DNA. Sonoporation using, e.g., the Sonitron 2000 system (Rich-Mar) can also be used for delivery of nucleic acids.

Additional exemplary nucleic acid delivery systems include those provided by Amaxa Biosystems (Cologne, Germany), Maxcyte, Inc. (Rockville, Md.), BTX Molecular Delivery Systems (Holliston, Mass.) and Copernicus Therapeutics Inc, (see for example U.S. Pat. No. 6,008,336). Lipofection is described in e.g., U.S. Pat. Nos. 5,049,386; 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424, WO 91/16024.

The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods of delivery include the use of packaging the nucleic acids to be delivered into EnGeneIC delivery vehicles (EDVs). These EDVs are specifically delivered to target tissues using bispecific antibodies where one arm of the antibody has specificity for the target tissue and the other has specificity for the EDV. The antibody brings the EDVs to the target cell surface and then the EDV is brought into the cell by endocytosis. Once in the cell, the contents are released (see MacDiarmid et al (2009) Nature Biotechnology 27(7):643).

The use of RNA or DNA viral based systems for the delivery of nucleic acids encoding engineered ZFPs take advantage of highly evolved processes for targeting a virus to specific cells in the body and trafficking the viral payload to the nucleus. Viral vectors can be administered directly to patients (in vivo) or they can be used to treat cells in vitro and the modified cells are administered to patients (ex vivo). Conventional viral based systems for the delivery of ZFPs include, but are not limited to, retroviral, lentivirus, adenoviral, adeno-associated, vaccinia and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system depends on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immunodeficiency virus (SIV), human immunodeficiency virus (HIV), and combinations thereof (see, e.g., Buchscher et al., J. Virol. 66:2731-2739 (1992); Johann et al., J. Virol. 66:1635-1640 (1992); Sommerfelt et al., Virol. 176:58-59 (1990); Wilson et al., J. Virol. 63:2374-2378 (1989); Miller et al., J. Virol. 65:2220-2224 (1991); PCT/US94/05700).

In applications in which transient expression is preferred, adenoviral based systems can be used. Adenoviral based vectors are capable of very high transduction efficiency in many cell types and do not require cell division. With such vectors, high titer and high levels of expression have been obtained. This vector can be produced in large quantities in a relatively simple system. Adeno-associated virus (“AAV”) vectors are also used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

At least six viral vector approaches are currently available for gene transfer in clinical trials, which utilize approaches that involve complementation of defective vectors by genes inserted into helper cell lines to generate the transducing agent.

pLASN and MFG-S are examples of retroviral vectors that have been used in clinical trials (Dunbar et al., Blood 85:3048-305 (1995); Kohn et al., Nat. Med. 1:1017-102 (1995); Malech et al., PNAS 94:22 12133-12138 (1997)). PA317/pLASN was the first therapeutic vector used in a gene therapy trial. (Blaese et al., Science 270:475-480 (1995)). Transduction efficiencies of 50% or greater have been observed for MFG-S packaged vectors. (Ellem et al., Immunol Immunother. 44(1):10-20 (1997); Dranoff et al., Hum. Gene Ther. 1:111-2 (1997).

Recombinant adeno-associated virus vectors (rAAV) are a promising alternative gene delivery systems based on the defective and nonpathogenic parvovirus adeno-associated type 2 virus. All vectors are derived from a plasmid that retains only the AAV 145 bp inverted terminal repeats flanking the transgene expression cassette. Efficient gene transfer and stable transgene delivery due to integration into the genomes of the transduced cell are key features for this vector system. (Wagner et al., Lancet 351:9117 1702-3 (1998), Kearns et al., Gene Ther. 9:748-55 (1996)). Other AAV serotypes, including AAV1, AAV3, AAV4, AAV5, AAV6 and AAV8, can also be used in accordance with the present invention.

Replication-deficient recombinant adenoviral vectors (Ad) can be produced at high titer and readily infect a number of different cell types. Most adenovirus vectors are engineered such that a transgene replaces the Ad E1a, E1b, and/or E3 genes; subsequently the replication defective vector is propagated in human 293 cells that supply deleted gene function in trans. Ad vectors can transduce multiple types of tissues in vivo, including non-dividing, differentiated cells such as those found in liver, kidney and muscle. Conventional Ad vectors have a large carrying capacity. An example of the use of an Ad vector in a clinical trial involved polynucleotide therapy for anti-tumor immunization with intramuscular injection (Sterman et al., Hum. Gene Ther. 7:1083-9 (1998)). Additional examples of the use of adenovirus vectors for gene transfer in clinical trials include Rosenecker et al., Infection 24:1 5-10 (1996); Sterman et al., Hum. Gene Ther. 9:7 1083-1089 (1998); Welsh et al., Hum. Gene Ther. 2:205-18 (1995); Alvarez et al., Hum. Gene Ther. 5:597-613 (1997); Topf et al., Gene Ther. 5:507-513 (1998); Sterman et al., Hum. Gene Ther. 7:1083-1089 (1998).

Packaging cells are used to form virus particles that are capable of infecting a host cell. Such cells include 293 cells, which package adenovirus, and ψ2 cells or PA317 cells, which package retrovirus. Viral vectors used in gene therapy are usually generated by a producer cell line that packages a nucleic acid vector into a viral particle. The vectors typically contain the minimal viral sequences required for packaging and subsequent integration into a host (if applicable), other viral sequences being replaced by an expression cassette encoding the protein to be expressed. The missing viral functions are supplied in trans by the packaging cell line. For example, AAV vectors used in gene therapy typically only possess inverted terminal repeat (ITR) sequences from the AAV genome which are required for packaging and integration into the host genome. Viral DNA is packaged in a cell line, which contains a helper plasmid encoding the other AAV genes, namely rep and cap, but lacking ITR sequences. The cell line is also infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV.

In many gene therapy applications, it is desirable that the gene therapy vector be delivered with a high degree of specificity to a particular tissue type. Accordingly, a viral vector can be modified to have specificity for a given cell type by expressing a ligand as a fusion protein with a viral coat protein on the outer surface of the virus. The ligand is chosen to have affinity for a receptor known to be present on the cell type of interest. For example, Han et al., Proc. Natl. Acad. Sci. USA 92:9747-9751 (1995), reported that Moloney murine leukemia virus can be modified to express human heregulin fused to gp70, and the recombinant virus infects certain human breast cancer cells expressing human epidermal growth factor receptor. This principle can be extended to other virus-target cell pairs, in which the target cell expresses a receptor and the virus expresses a fusion protein comprising a ligand for the cell-surface receptor. For example, filamentous phage can be engineered to display antibody fragments (e.g., FAB or Fv) having specific binding affinity for virtually any chosen cellular receptor. Although the above description applies primarily to viral vectors, the same principles can be applied to nonviral vectors. Such vectors can be engineered to contain specific uptake sequences which favor uptake by specific target cells.

Gene therapy vectors can be delivered in vivo by administration to an individual patient, typically by systemic administration (e.g., intravenous, intraperitoneal, intramuscular, subdermal, or intracranial infusion) or topical application, as described below. Alternatively, vectors can be delivered to cells ex vivo, such as cells explanted from an individual patient (e.g., lymphocytes, bone marrow aspirates, tissue biopsy) or universal donor hematopoietic stem cells, followed by reimplantation of the cells into a patient, usually after selection for cells which have incorporated the vector.

Vectors (e.g., retroviruses, adenoviruses, liposomes, etc.) containing nucleases and/or donor constructs can also be administered directly to an organism for transduction of cells in vivo. Alternatively, naked DNA can be administered.

Administration is by any of the routes normally used for introducing a molecule into ultimate contact with blood or tissue cells including, but not limited to, injection, infusion, topical application and electroporation. Suitable methods of administering such nucleic acids are available and well known to those of skill in the art, and, although more than one route can be used to administer a particular composition, a particular route can often provide a more immediate and more effective reaction than another route.

Vectors suitable for introduction of polynucleotides described herein include non-integrating lentivirus vectors (DLV). See, for example, Ory et al. (1996) Proc. Natl. Acad. Sci. USA 93:11382-11388; Dull et al. (1998) J. Virol. 72:8463-8471; Zuffery et al. (1998) J. Virol. 72:9873-9880; Follenzi et al. (2000) Nature Genetics 25:217-222; U.S. Patent Publication No 2009/054985.

Pharmaceutically acceptable carriers are determined in part by the particular composition being administered, as well as by the particular method used to administer the composition. Accordingly, there is a wide variety of suitable formulations of pharmaceutical compositions available, as described below (see, e.g., Remington's Pharmaceutical Sciences, 17th ed., 1989).

It will be apparent that the nuclease-encoding sequences and donor constructs can be delivered using the same or different systems. For example, a donor polynucleotide can be carried by a plasmid, while the one or more nucleases can be carried by a AAV vector. Furthermore, the different vectors can be administered by the same or different routes (intramuscular injection, tail vein injection, other intravenous injection, intraperitoneal administration and/or intramuscular injection. The vectors can be delivered simultaneously or in any sequential order.

Formulations for both ex vivo and in vivo administrations include suspensions in liquid or emulsified liquids. The active ingredients often are mixed with excipients which are pharmaceutically acceptable and compatible with the active ingredient. Suitable excipients include, for example, water, saline, dextrose, glycerol, ethanol or the like, and combinations thereof. In addition, the composition may contain minor amounts of auxiliary substances, such as, wetting or emulsifying agents, pH buffering agents, stabilizing agents or other reagents that enhance the effectiveness of the pharmaceutical composition.

### Parkinson's Disease, Cancer and LRRK2

As described above, mutations in LRRK2 are found in both familial and sporadic cases of PD. The instant invention describes methods and compositions that can be used to introduce or repair such mutations.

In particular, specific mutations encoded by mutant LRRK2 genes that have been proven shown to be pathogenic in the development of PD include Y1699C, R1441C, R1441H, R1441H, I1371V, Y1699G, G2019S, I2020T, and G2385R. Mutations within LRRK2 that are potentially pathogenic include E334K, Q1111H, I1192V, I1122V, S1228T, A1442P, L1719F, and T23561. Those mutations that are associated with an increased risk of developing PD are R1628P and G2385R (see Kumari, ibid). Thus, the methods and compositions of the instant invention are useful for repairing such mutations in LRRK2, and are useful for developing cell and transgenic animal models to study the intracellular pathology associated with LRRK2 mutations and for studying the whole organism consequences of these mutations.

Mutations in LRRK2 may also be implicated in some types of cancers, including but not limited to, melanomas, renal and thyroid cancers. Thus, tools designed to knock out or knock in specific LRRK2 mutations such as the G2019S LRRK2 mutation in cancer models will be useful in furthering an understanding of the underlying biology and in the development of specific drug therapies. Further, specific nucleases targeted to a specific LRRK2 mutation can be employed to knock out or correct the mutation. Nucleases can also be used to cause the insertion of an LRRK2 mutation-specific tag in order to develop cell lines for the investigation of LRRK2 mutation specific therapeutics.

Additionally, cells, cell lines and transgenic animals as described herein are useful for drug development. Such cells and animals may reveal phenotypes associated with a particular mutation (e.g. LRRK2 G2019S alterations) or with its correction, and may be used to screen drugs that will interact either specifically with the mutation(s) or mutant proteins in question, or that are useful for treatment of the disease in an afflicted animal. Therapeutically, iPSCs can be derived ex vivo from a patient afflicted with a known genetic mutation associated with PD, and this mutation can be corrected using ZFN- or TALEN-mediated gene correction. The corrected iPSC can then be differentiated into dopaminergic neurons and reimplanted into the patient. Alternatively, the ZFNs may be introduced into the patient's cells in vivo for in situ gene correction. These cell lines can also provide tools to investigate the effects of specific mutations patient-specific iPS cell lines that are only different by the disease-causing mutation, thus representing a ‘genetically virtually identical’ control cell line. The resulting isogenic panel of iPSCs that carry different allelic forms of LRRK2 at the endogenous locus provides a genetic tool for repair of disease-specific mutations, drug screening and discovery, and disease mechanism research.

The availability of patient-specific iPS cell lines with both repaired and induced mutations and their isogenic controls are also useful in a wide-variety of medical applications, including but not limited to, the study of mechanisms by which these mutations cause disease to translating these “laboratory cures” to treatments for patients who actually manifest disease induced by these mutations.

The following Examples relate to exemplary embodiments of the present disclosure in which the nuclease comprises a zinc finger nuclease (ZFN). It will be appreciated that this is for purposes of exemplification only and that other nucleases can be used, for instance homing endonucleases (meganucleases) with engineered DNA-binding domains and/or fusions of naturally occurring of engineered homing endonucleases (meganucleases) DNA-binding domains and heterologous cleavage domains or TALENs.

### EXAMPLES

### Example 1

### Nucleases Specific for LRRK2

ZFN pairs targeting the human LRRK2 gene in proximity of the rs34637584 SNP were used to test the ability of these ZFNs to induce DSBs at a specific target site. The amino acid sequence of the recognition helix region of the indicated ZFNs are shown below in Table 2 and their target sites shown in Table 3 (DNA target sites indicated in uppercase letters; non-contacted nucleotides indicated in lowercase). The Cel-I assay (Surveyor™, Transgenomics. Perez et al, (2008) Nat. Biotechnol. 26: 808-816 and Guschin et al, (2010) Methods Mol Biol. 649:247-56), was used where PCR-amplification of the target site was followed by quantification of insertions and deletions (indels) using the mismatch detecting enzyme Cel-I (Yang et al, (2000) Biochemistry 39, 3533-3541) which provides a lower-limit estimate of DSB frequency. Two days following transfection of the ZFN expression vector, genomic DNA was isolated from K562 cells using the DNeasy kit (Qiagen). In these experiments, all ZFN pairs were ELD/KKR FokI mutation pairs (described above). In addition, ZFNs specific for mutant LRRK2 genes were also developed. ZFNs with preference for the 6055G>A gene mutant (resulting in a G2019S mutant LRRK2 protein) and are also shown below in Tables 2 and 3.

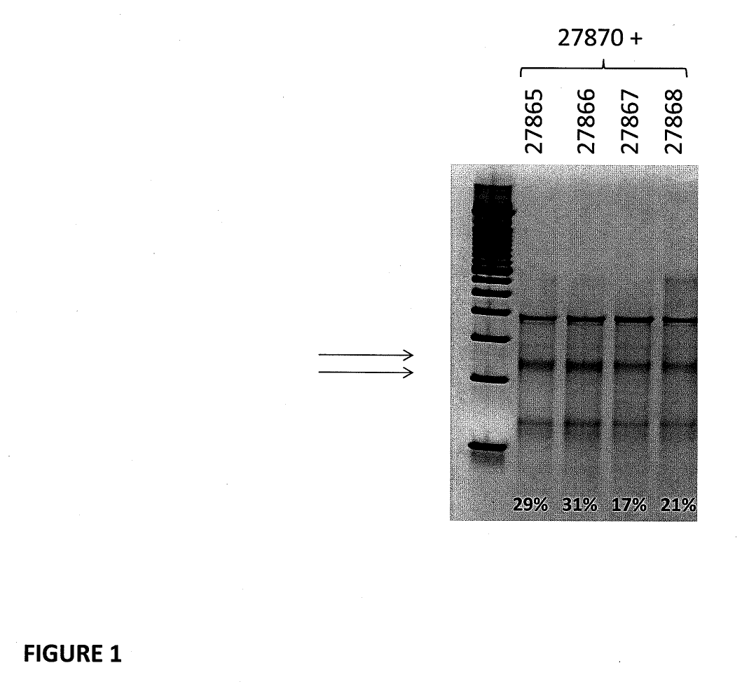

Results from the Cel-I assay are shown in FIG. 1, and demonstrate that the ZFNs shown below are capable of inducing cleavage at their respective target sites.

TABLE 2
LRRK2-specific ZFNs

| ZFN name | F1 | F2 | F3 | F4 | F5 | F6 |
|---|---|---|---|---|---|---|
| SBS 27870 | QSGDLTR | RNDILAS | DRSNLSR | LRQDLKR | TSGNLTR | QSNQLRQ |
| | (SEQ ID | (SEQ ID | (SEQ ID | (SEQ ID | (SEQ ID | (SEQ ID |
| | NO: 1) | NO: 2) | NO: 3) | NO: 4) | NO: 5) | NO: 6) |
| SBS 27865 | QSGHLSR | RSDSLSV | QSSDLRR | QSGDLTR | RKDPLKE | N/A |
| | (SEQ ID | (SEQ ID | (SEQ ID | (SEQ ID | (SEQ ID | |
| | NO: 7) | NO: 8) | NO: 9) | NO: 1) | NO: 10) | |
| SBS 27866 | QSGHLSR | RSDSLSV | QSSDLRR | QSGDLTR | RRDPLIN | N/A |
| | (SEQ ID | (SEQ ID | (SEQ ID | (SEQ ID | (SEQ ID | |
| | NO: 7) | NO: 8) | NO: 9) | NO: 1) | NO: 11) | |
| SBS 27867 | QSGHLAR | RKWTLQG | QSSDLSR | QWSTRKR | RSDALTQ | N/A |
| | (SEQ ID | (SEQ ID | (SEQ ID | (SEQ ID | (SEQ ID | |
| | NO: 12) | NO: 13) | NO: 14) | NO: 15) | NO: 16) | |
| SBS 27868 | QSGHLAR | RKWTLQG | QSGDLTR | QSGDLTR | RKDPLKE | N/A |
| | (SEQ ID | (SEQ ID | (SEQ ID | (SEQ ID | (SEQ ID | |
| | NO: 12) | NO: 13) | NO: 1) | NO: 1) | NO: 10) | |
| SBS 30886 | QSGHLSR | RSDSLSA | QSGDLTR | QSGDLTR | RKDPLKE | N/A |
| | (SEQ ID | (SEQ ID | (SEQ ID | (SEQ ID | (SEQ ID | |
| | NO: 7) | NO: 20) | NO: 1) | NO: 1) | NO: 10) | |
| SBS 30888 | QSGDLTR | QWGTRYR | DRSNLSR | LRQDLKR | TSGNLTR | QSNQLRQ |
| | (SEQ ID | (SEQ ID | (SEQ ID | (SEQ ID | (SEQ ID | (SEQ ID |
| | NO: 1) | NO: 21) | NO: 3) | NO: 4) | NO: 5) | NO: 6) |

TABLE 3
Target sites for LRRK2-specific ZFNs

| ZFN Name | Target Site |
|---|---|
| SBS 27870 | gcAAAGATtGCTGACtACGGCAttgctc |
| | (SEQ ID NO: 17) |
| SBS 27865 | tgATGGCAGCATTGGGAtacagtgtgaa |
| | (SEQ ID NO: 18) |
| SBS 27866 | tgATGGCAGCATTGGGAtacagtgtgaa |
| | (SEQ ID NO: 18) |
| SBS 27867 | tgATGGCAGCATTGGGAtacagtgtgaa |
| | (SEQ ID NO: 18) |
| SBS 27868 | tgATGGCAGCATTGGGAtacagtgtgaa |
| | (SEQ ID NO: 18) |
| SBS 30886 | tgATGGCAGCATTGGGAtacagtgtgaa |
| | (SEQ ID NO: 18) |
| SBS 30888 | gcAAAGATtGCTGACtACAGCAttgctc |
| | (SEQ ID NO: 22) |

### Example 2

### Insertion of a 6055G>A Mutation into a Wild Type LRRK2 Allele

It would be advantageous to be able to create a mutant allele in the LRRK2 in any desired cell type to allow for study of the mutation in a variety of genotypic backgrounds.

Accordingly, the LRRK2-specific ZFNs were used to induce targeted integration of the sequences encoding this mutation within wild type cells using a donor DNA including the mutation. For donor, 1000 bp of a PCR product generated by performing a PCR reaction on patient DNA carrying the mutation was used, and is shown below, where the mutation is indicated in bold and underlined font:

(SEQ ID NO: 19)
5′AGCTGAGCTAAACCTCTATGTGGTTTTAGGAAAATCAAAACTATTAAA
TAAATGGCAAGTACAACAAAATCCCATCAATTCTTATTTAACATACTTAC
ATTTTGAAATAGTTAAAATATTCATATGATCATTGAGAGAATTCAGAATT
GCCTTTAAGTAATTGTTCACATATACAAAAGAAAAGTCTCCAAAAATTGG
GTCTTTGCCTGAGATAGATTTGTCTTAAAATTGAAATCATTCACTTATCA
GATTTGACCCTTTTTTAAAGCATAACTTTGCTGTGTAATATTAGACTTAT
ATGTTTTGATTTCCTTCTACAATATCTCTTAACTTTAAGGGACAAAGTGA
GCACAGAATTTTTGATGCTTGACATAGTGGACATTTATATTTAAGGAAAT
TAGGACAAAAATTATTATAATGTAATCACATTTGAATAAGATTTCCTGTG
CATTTTCTGGCAGATACCTCCACTCAGCCATGATTATATACCGAGACCTG
AAACCCCACAATGTGCTGCTTTTCACACTGTATCCCAATGCTGCCATCAT
TGCAAAGATTGCTGACTACAGCATTGCTCAGTACTGCTGTAGAATGGGGA
TAAAAACATCAGAGGGCACACCAGGTAGGTGATCAGGTCTGTCTCATAAT
TCTATCTTCAGGATGGATAACCACTGACCTCAGATGTGAGTTCAGAAGAG
TCAAAAGGAAAACAGAGTCTATCACATTGTGAACAGAGGTTTATTTTGTG
AAAAAATGCAAGCATCACATTGTGATTTTTATCATTGTATTTTGTAGGAA
AAAAACAATTGATGTAATTTTTCAGGGCAAAAACTGAATAAAAAGAAGAG
AATGTTTGATATCAAGTTATATGTTTTAAAGTTAGATTTGTAGATTCTTT
AGATACTCTAGAGGTCATAAAAAGTAACAGCAAAAACTTTAGTCTAGGTA
TTGTTGGCACTTGTGAGGCAAATCAAATTCAGGTCCACAAATTCTTTTTC
3′

The LRRK2-specific ZFNs were nucleofected into K562 cells along with varying amounts of donor (from 0.5 μg-2.0 μg) according to standard protocol as described above. DNA was then isolated from the transfectants and used for a PCR reaction where a first PCR reaction was done to amplify the region that encompasses the nucleotide 6055; this reaction was then diluted and subjected to another PCR yielding an approximately 320 bp product that was then subjected to SfcI digestion which exclusively recognizes the mutant sequence and creates a faster moving product on a gel.

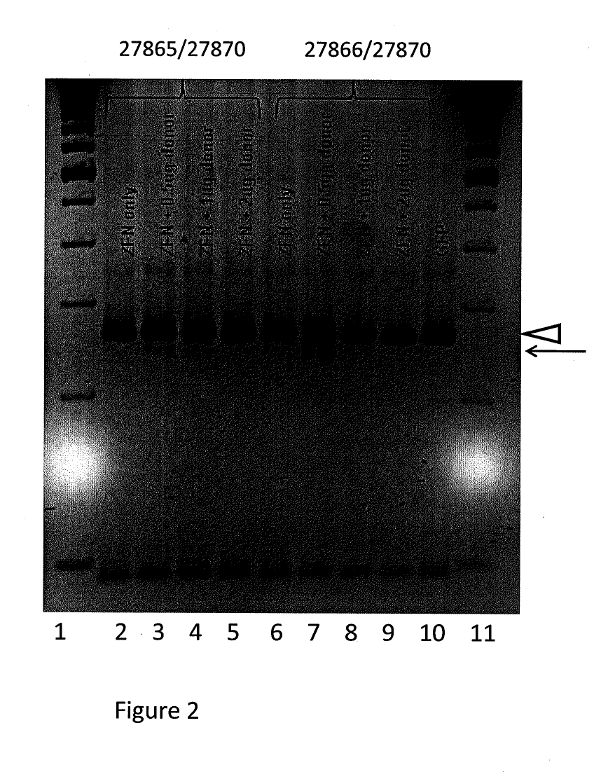

The results are depicted in FIG. 2. Controls included nucleofection with the ZFNs only (lanes 2 and 6) and nucleofection with a plasmid encoding GFP (lane 10). The mutant digestion bands are evident in lanes 3, 4 and 7, (indicated with an arrow) demonstrating that these clones now carry the 6055 G>A mutation associated with the G2019S LRRK2 mutant allele. The band from the wild type gene is indicated with an open triangle.

### Example 3

### Isolation of Single-Cell Clones from Pools of Patient Cells Modified with ZFN

To test if we could isolate single cell clones from the patient fibroblast pools, fibroblasts from patients heterozygous for 6055G>A, (encoding the disease associated G2019S mutation in the expressed protein) in the LRRK2 gene were cultured and nucleofected with LRRK2-specific ZFNs and donor DNA bearing a wild type 1 kb genomic fragment. Pools were cultured for 3 days, assayed for Cel-1 activity as described above, and then single-cell cloned. Thirty-six clones were cultured and their genomic DNA was extracted for genotype analysis. A PCR reaction was performed to amplify the LRRK2 locus and then the product was subjected to BsrDI digestion. The BsrDI restriction enzyme recognizes the wild type sequence in the middle of the ZFN cleavage site: a disruption of this site will most like occur in the presence of an NHEJ event following cleavage at that location.

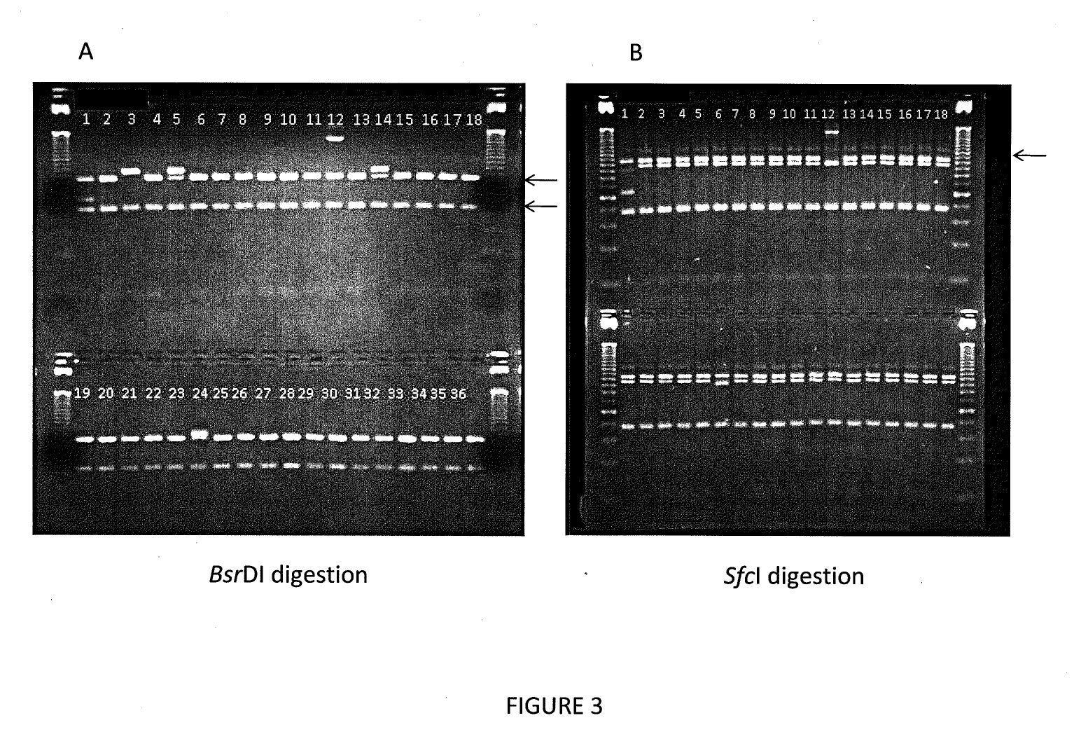

The results from both these digestions are shown in FIG. 3. For the BsrDI digestion (FIG. 3A) arrows indicate the expected products of the digest. Any deviation from these bands indicates that NHEJ has occurred. Furthermore, clones were PCR amplified and digested with the SfcI enzyme, which only cuts the mutant allele. A pattern showing only the wild type band (upper band from the doublet highlighted by the arrow in FIG. 3B, SfcI resistant fragment) may indicate a gene correction event.

### Example 4

### Production of iPSC from ZFN Modified Patient Fibroblasts

We generated iPSCs from the ZFN-modified fibroblast pools. Fibroblast pools were cultured and then transduced with retroviral vectors expressing Oct4, Sox2, Klf4 and c-myc (Takahashi and Yamanaka, Cell (2006) 126(4):663-76) using standard protocols. In particular, fibroblast pools were generated from 14 patients heterozygous for the LRRK2 G2019S mutation and from three patients homozygous for the mutation. Twenty iPSC clones were derived, and then were genotyped similarly by PCR and enzymatic digestion as described for the fibroblast clones in Example 3.

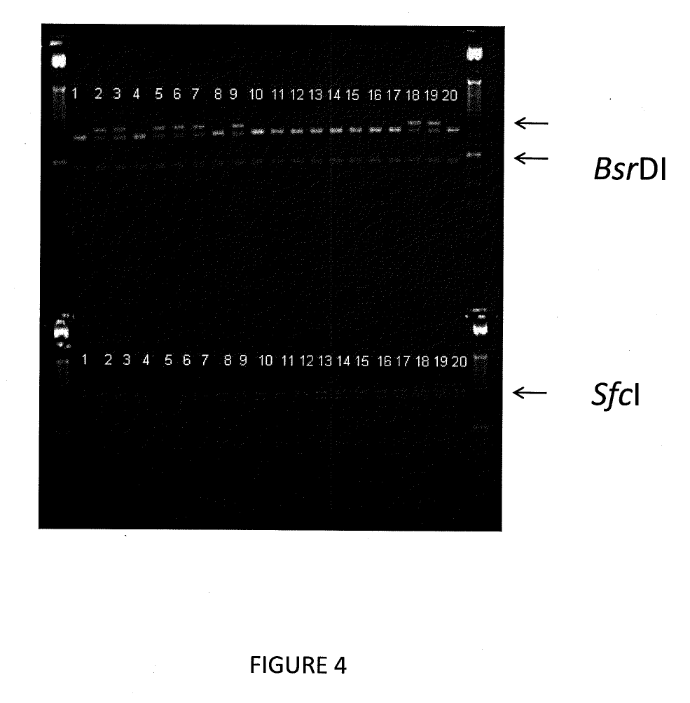

The results are presented in FIG. 4, which shows the results of the SfcI and BsrDI digestions. Arrows indicate the expected products in the BsrDI digestion and the wild type band in the SfcI digestion (as in FIG. 3). In addition, these lines were characterized for pluripotency and were found to be karyotypically normal.

Patient-derived iPSCs were also evaluated for their ability to generate dompaminergic neurons using a technique involving embryoid body formation followed by attachment of embryoid bodies and isolation of neuronal rosettes expressing Pax6 and counterstained with Hoechst. Pure rosette cultures were isolated and expanded as neural stem cells (NSCs) with expressing Nestin and Sox 1 in over 90% of the cells. The NSCs can be expanded for up to ten passages and cryopreserved.

For dopaminergic differentiation the NSCs were cultured in Neurobasal medium supplemented with sonic hedgehog (Shh) (200 ng/ml) and FGF8 50 ng/ml for 10 days followed by withdrawal of Shh and FGF8 and replacement of BDNF (20 ng/ml), GDNF (20 ng/ml), and dcAMP (1 mM) for 20-25 days. After 30 days, cells showed a high yield of β-tubulin type III, a marker for an early neuronal phenotype. At day 35, tyrosine hydroxylase (TH) was detected in 15-20% of the cells as well as a high yield of microtubule associated protein 2 (MAP2), a postmitotic mature neuronal marker. The yield of midbrain dopaminergic (mDA) neurons was further increased by culturing these iPSC-derived neurons under low oxygen conditions (3-5% oxygen). See, e.g., Studer et al. J. Neurosci. (2000) 20:7377-7383.

Thus, NSCs and TH-positive neurons were derived from normal and LRRK2 positive clonal iPSC lines. These cells were further characterized for other specific mDA markers (i.e. Pitx3, Nurr-1, Lmx1A, AADC, VMAT-2 and Girk2). Data showed that during a time course the mDA neurons from a patient carrying the LRRK2 G2019S on both alleles showed an increase in markers of stress response genes and an increase in alpha-synuclein protein expression compared to control lines exhibiting an early functional phenotype, both of which could be characteristic of PD pathology. Mitochondrial function and lysosomal abnormalities are also evaluated in these iPS-derived mDA neurons.

### Example 5

### ZFN Modification of Patient Derived Fibroblasts and Patient-Derived iPSCs

ZFNs can be used cleave the LRRK2 gene in patient-derived fibroblasts or patient-derived iPSCs.

To assay ZFN-modification in patient-derived fibroblasts, the cells were treated with ZFNs as described above. Following ZFN treatment, a 346-bp region encompassing the ZFN cleavage site was PCR-amplified from ZFN-transfected cell pools to generate a mixture of unmodified as well as modified amplicons (derived from NHEJ-mediated imperfect repair of the DNA break); denaturing and reannealing of this mixture results in mismatches between heteroduplexes of the unmodified and modified alleles, which create distortions in the DNA that are recognized and cleaved by Cel-1 nuclease.

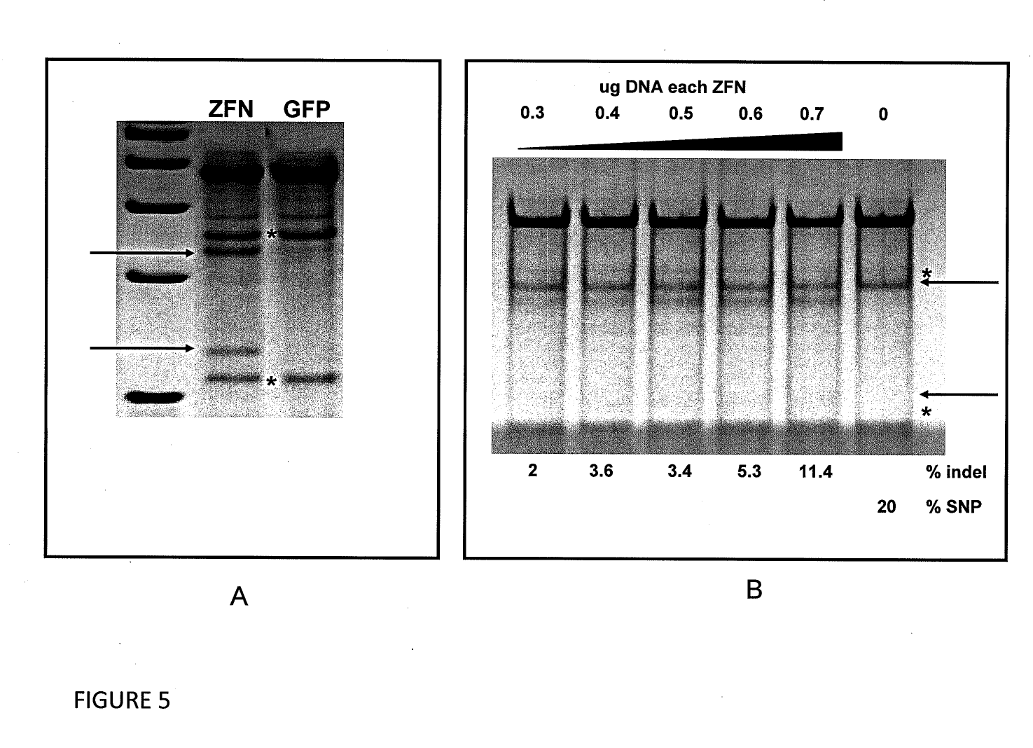

As shown in FIG. 5A, LRRK2-targeted ZFNs efficiently modified (˜16%) patient-derived fibroblasts. Sequencing analysis of 7 clones revealed 3 clones that contain frameshift mutations of the G2019S encoding allele, thus disrupting the translation of the protein. These results indicate that ZFN-induced cleavage followed by NHEJ occurred in these cells.

Following LRRK2 gene modification in patient-derived fibroblasts, iPSCs can be derived from ZFN/donor-transfected fibroblasts pool. This approach provides an alternative path to corrected iPS cells, because fibroblasts are easier to maintain in cell culture, they can be transfected with significantly higher efficiency (>90% using nucleofection). In this approach, patient-derived fibroblasts are nucleofected with LRRK2 ZFN expression vectors and the donor construct. Cel-1 nuclease assay will be performed on a sub-population of transfected cells to confirm that efficient cleavage by ZFNs has occurred at the LRRK2 locus. The transfected pool is used for iPSC derivation; the resultant iPSC clones are subject to genotype analysis as described above, clones with 2 wt LRRK2 alleles, as well as those with frameshift mutation on the G2019S allele and intact wt allele, are identified and their pluripotency verified before additional functional characterization. However during iPS generation, mosaic clones that contain different ZFN-mediated events in the resulting clonal line may be obtained, thus requiring additional subcloning but may give better overall efficiency of gene modification. Also alternatively, we can use non-integrating lentiviral vector to deliver both the ZFNs and the donor to patient-derived iPS cells.

In addition, ZFN modification of LRRK2 was performed in patient-derived iPSCs. DNA sequences (e.g., plasmids) encoding LRRK2 ZFNs, the green fluorescent protein (GFP) and a donor DNA construct were co-delivered by nucleofection to patient-derived iPSCs that are heterozygous for the G2019S mutation. The donor construct contains 1 kb of wild type (wt) LRKK2 genomic sequence (500 bps in each direction of the mutation). In a subpopulation of cells that contain ZFN-mediated DSB, HDR the donor was used as a template to repair the DSB as well as the mutation; the majority of the cells will use NHEJ to resolve the DSB. Transfected (GFP-positive) cells are enriched through fluorescence-based cell sorting and replated on MEF feeder layers for single-cell cloning.

Genomic DNA was isolated from clones, the region of interest are amplified by PCR and subject to digest by restriction enzymes SfcI and BsrDI. SfcI only cleaves unmodified G2019S encoding allele; while BsrDI site is destroyed if deletion or insertion has occurred at the site of DSB (indicating NHEJ). Single cell-derived clones that contain 2 wt alleles (gene correction) lose the SfcI site and retain BsrDI site on both alleles, while clones that contain intact wild type allele and disrupted G2019S allele retain BsrDI on one allele.

Clones meeting these criteria were subject to sequencing analysis to confirm the presence of 2 wild type LRRK2 alleles for G2019S-corrected cells, and to identify those having frameshift mutations on the mutant allele and unmodified wild type allele. Before further functional characterization of gene-corrected or G2019S-disrupted iPSC clones, cytogenetic analysis is performed to confirm normal karyotype, and their pluripotent state verified by expression of the pluripotency markers (OCT4, NANOG and SOX2), as well as ability to form all three developmental germ layers in teratoma-formation assays.

As described above, the LRRK2-specific ZFNs were transfected into patient derived iPSC. The patient cells were heterozygous for the G2019S mutation. An increasing concentration of ZFN expression plasmids were used to see if there was a concomitant increase in gene modification as measured by the Cel I assay. The results demonstrated that the ZFNs were able to induce greater than 11% gene modification (see FIG. 5B).

The assay was performed as follows. Cell culture and transfection: The iPSC were grown in mTeSR®1 medium (STEM CELL™ Technologies) on Matrigel coated dishes (BD BioCoat™, BD Biosciences) and the media changed daily. One day before transfection, 10 uM of Rho-associated protein kinase (ROCK) Inhibitor (Calbiochem Y-27632) was added to the culture. Transfection was carried out with the Amaxa (Lonza) 96-well shuttle using P4 Primary Cell 96-well Nucleofector Kit following manufacturer's instructions: 2e5 cells were transfected with ZFN-encoding plasmid DNA or mRNA in the presence of donor DNA to trigger homologous recombination and gene correction.

To determine the level of gene modification, a Cell assay was performed using the following primers:

LRRK2Cel111615821F
(SEQ ID NO: 23)
5′ AGG GAC AAA GTG AGC ACA GAA
LRRK2Cel111650122R
(SEQ ID NO: 24)
5′ GAG GTC AGT GGT TAT CCA TCC T

The Cel I assay was performed using 1-3 μl of a 1/1000 dilution of the PCR described above.

Next, a donor was designed to correct the mutation. This donor was designed with a silent mutation such that the zinc finger DNA binding domains would have a weaker affinity for the donor sequence and would be less frequently cleaved by the ZFNs. In addition, this mutation also introduced an AciI restriction site which allowed the tracking of the recombined allele. The donor was constructed by amplifying the wild type allele and then inserting the mutation described above. The sequence, ZFN binding sites and diagnostic restriction sites (in italics) are shown below:

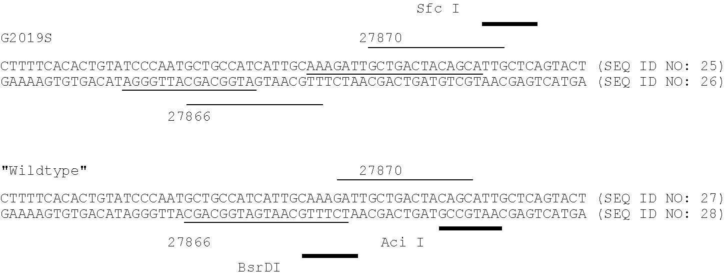

In the G2019S sequence, the mutation in the sequence is shown in bold. In the “wild type” donor, the mutations made to insert an AciI restriction site and decrease affinity for the 27870 ZFN are shown in bold. A second ZFN pair, termed 30886 and 30888, was also constructed (shown in Tables 2 and 3) with an increased ability to cut the mutant G2019S sequence. To analyze the insertion of the donor, RFLP analysis was performed to assess the presence or absence of the AciI restriction site. The RFLP assay was carried out as follows. Cells were lysed in quick extract and PCR was performed using the following primers:

LRRK2-2 outout F
(SEQ ID NO: 29)
5′ CTC TTT GCC TGA AAT GTC CAA CTC TAG
LRRK2-2 out out R
(SEQ ID NO: 30)
5′ CAA TCA GCA AGA TGA TGT ATA GCC ACC TG

These 2 kb PCR products were purified and digested with the AciI enzyme to assess the HR/RFLP that occurred in the transfected cells, and analyzed via agarose gel electrophoresis.

Clones were obtained with the desired gene modifications as follows: After transfection, the iPSC pools were amplified and analyzed for gene modification by the Cell and RFLP assays. Cell pools were treated with Accutase (ACCUTASE™, Millipore) to achieve a suspension of single cells. Then, Matrigel-coated 96-well plates were prepared, and cells were counted and 2 cells per well were seeded in mTeSR®1 with ROCK inhibitor. Medium was changed every second day for a week and then daily. Half of every single cell clone was used for subsequent genotyping as follows: cells were lysed in quick extract and submitted to nested PCR as described above, before proceeding to the genotyping. Genotyping by digest: PCR products from each clones were purified and digested with AciI. Also, as described for the genotyping of the fibroblast clones, they were digested with BsrDI (a disruption of this restriction enzyme site indicates an NHEJ event) and SfcI (this enzyme only cleaves a mutant G6055A site, so the absence of cleavage indicates gene correction). Genotyping by sequencing: For genotyping by sequencing, the PCR product was Topo cloned (Invitrogen) and sequenced using the LRRK2Cel111615821F primer.

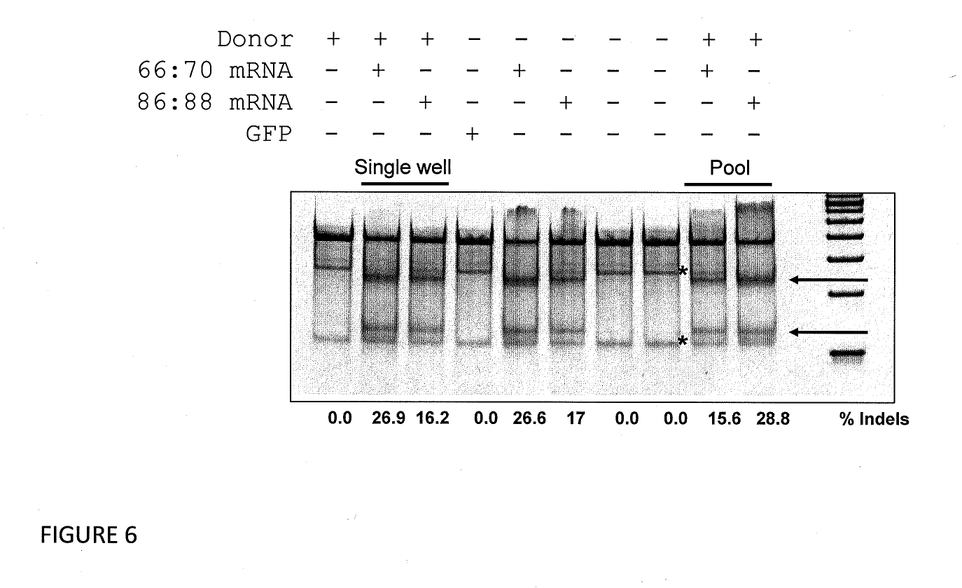

The iPSCs derived from the heterozygous patient described above were transfected with the two pairs of ZFNS, the 27866/70 pair, which is more specific for the wildtype sequence, and the 30886/88 pair, which is more specific for the G2019S mutant sequence. In the presence of the donor construct, the ZFN pairs were analyzed for donor insertion from either single clone cultures, or from transfectant cell pools. Evidence of gene modification was found using both ZFN pairs and in the presence or absence of donor (see FIG. 6).

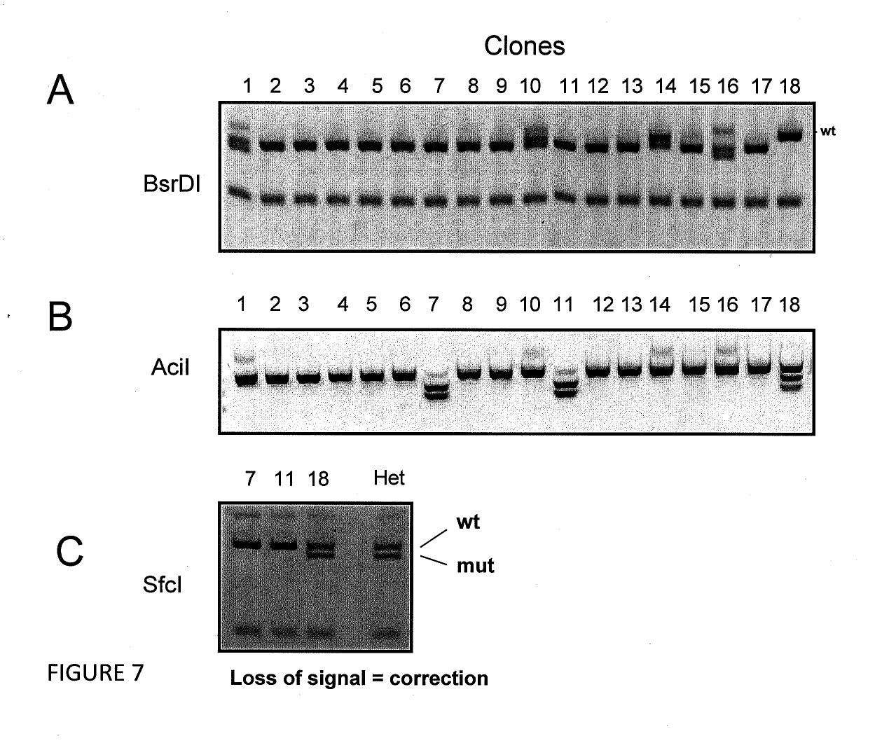

Individual clones treated with the 27866/70 pair (“66-70”) or the 30886/88 (“86-88”) pair and donor were examined for the presence of the donor sequences and to confirm gene correction at the G2019S mutation site. In addition, the clones were examined to determine if donor integration had occurred through NHEJ or HR. DNA was isolated as described previously and subject to PCR to amplify the insertion region as described above. The amplified DNA was then subject to three separate digestions with BsrD1, AciI and SfcI, (see above for locations of restriction sites). The BsrD1 restriction enzyme cuts in between the ZFN binding sites, and the absence of cleavage by this enzymes demonstrates insertion of donor sequences. AciI cleavage is diagnostic of gene correction by FIR, and lack of SfcI cleavage is diagnostic for gene correction of the G2019S mutation. The restriction digests demonstrated that the donor had been inserted and that the G2019S mutation had been corrected in the iPSC derived from patient fibroblasts (see FIG. 7). In the experiment, the recovery of the different types of gene modification outcomes found in the clones analyzed (47 for the 66-70 transfection, 100 for the 86-88 transfection) are shown below in Table 4:

TABLE 4
Analysis of Genetic Outcomes

| | NHEJ | | NHEJ | | HR | | HR | |
|---|---|---|---|---|---|---|---|---|
| | (Monoallelic) | | (Biallelic) | | (Monoallelic) | | (Biallelic) | |
| | 66-70 | 86-88 | 66-70 | 86-88 | 66-70 | 86-88 | 66-70 | 86-88 |
| # of | 9 | 46 | 3 | 17 | 3 | 2 | 3* | 14 |
| clones | | | | | | | | |
| % of | 19 | 46 | 6.4 | 17 | 6.4 | 2 | 6.4 | 14 |
| total | | | | | | | | |

*One clone had an additional NHEJ on corrected allele.

These results demonstrate that the ZFNs are able to cause specific gene correction in iPSC derived from patient fibroblasts.

### Example 6

### Generation of PD Models

Thus, the compositions and methods described herein can be used to generate model systems for the study of PD. For example, patient-derived iPSCs with corrected or disrupted G2019S allele provide cell models to determine whether the G2019S mutation is necessary for developing PD-related phenotypes, and to characterize the relative contribution of the G2019S to PD phenotype.

To mitigate concerns that phenotypes observed in downstream characterization are due to variations intrinsic to the iPSC generation process (e.g. random integration of the reprogramming cassette), correction and disruption of the G2019S mutation is performed in a minimum of two independent iPSC lines derived from the same patient; and the same process carried out on iPSCs derived from 3 unrelated patients that carry the mutation, thereby providing isogenic cell models for studying the role of G2019S mutation in the context of different genetic background.

For certain models, the LRRK2 ZFNs are used to introduce DSBs to the LRRK2 locus in iPSCs derived from normal subjects, and HDR invoked for de novo creation of monoallelic G2019S mutation. The iPSCs are altered as described above, except the cells are derived from LRRK2-normal subjects and the donor construct contains a nucleotide sequence that introduces the G2019S mutation. The desired clone will contain SfcI site on one allele and BsrDI on both alleles, clones with the expected digest pattern will be sequenced to verify the engineered mutation.

The resultant iPS cell lines will provide powerful evidence whether the G2019S mutation is sufficient for producing PD-related phenotypes in terms of increased kinase activity and ultimately pathological manifestation of PD in dopaminergic neurons derived from iPS cells.

The impact of the ZFN-mediated gene editing on the LRRK2 protein and LRRK2 kinase activity in iPS cells is also assayed, particularly by evaluating accumulation of LRRK2 in ZFN-modified cells as compared to the corresponding unmodified cells and/or wild-type cells, using immunoblot analysis. LRRK2 kinase activity in edited iPS cells is also evaluated by assessing binding to a novel and specific peptide sequence-Nictide (Nichols et al. (2010) Biochem. J. 430(3):393-404). Briefly, the amount of LRRK2 kinase activity in these cells is evaluated by immunoprecipitation of endogenous LRRK2 from wild-type, G2019S het and ZFN mediated wild-type reversions, followed by assaying the activity against Nictide in immune-complex kinase assays. Alternatively, LRRK2 kinase activity is determined by monitoring feedback phosphorylation event on Serines 910 and 935, for example using phospho-specific antibodies against phosphoserine 910 and phosphoserine 935. See, e.g. Nichols et al. (2010) Biochem. J. 430(3):393-404; Dzamko et al. (2010) Biochem. J. 430(3):405-413. Other antibodies targeted to LRRK2 constitutive phosphorylation sites as well as against autophosphorylation sites can provide an additional readout of LRRK2 kinase activity. Furthermore, reagents can be used to detect the modification of a direct target of LRRK2. Having isogenic control cell lines should add great precision to these experiments.

### Example 7

### Evaluation of a PD-Related Phenotype in ZFN-Modified Cell Lines in Differentiated Midbrain Dopaminergic Neurons

Midbrain dopaminergic (mDA) neurons that show characteristics of A9 mDA neurons of the substantia nigra are derived from LRRK2 iPSCs essentially as described in Example 4. These mDA neurons are evaluated for the expression (RT-PCR and IHC) of other midbrain-consistent markers. See, e.g., Abeliovich et al. (2007) Dev Biol. 304:447-454; Gale et al. (2008) Mol Brain. 1:8. Neuronally differentiated iPS cells are examined for signs of spontaneous pathology starting with simple measures of mDA cell abundance (% DA neurons after differentiation) and general morphological attributes such as inclusion bodies, or dystrophic neuritis and functional dopamine release measured by HPLC. See, e.g., Liu et al. (2009) Brain Res Bull 80:62-68; Papanikolaou et al. (2008) Stem Cells Dev. 17:157-172. As described above in Example 4, increases in expression of stress response genes and an increase in alpha-synuclein protein expression in iPSC-derived neurons derived from LRRK2 G2019S carriers have been observed.

In addition, mDA neurons from the generated panel of ‘repaired’, disrupted, and created G2019S mutation and are compared to each other and to the unmodified “original” iPSC lines. Several clones from the same lines will be tested as well as lines from unrelated LRRK2 carriers.

All generated cell lines are compared for molecular signs of pathology of PD, which include biochemical markers of oxidative stress, α-synuclein overexpression, and possibly mitochondrial and lysosomal dysfunction. The proposed assays for disease-associated mechanisms include apoptosis (TUNEL, caspase activation), necrosis (CytoTox-Glo), 3) oxidative stress (glutathione, ROS and 4-HNE), 4) mitochondrial dysfunction (ATP-lite: ATP production, membrane potential, mitochondrial content). Protein aggregation of α-synuclein will be assessed with antibodies against total, phosphorylated and nitrated α-synuclein to detect aggregates, inclusion bodies, and neuritic pathology.

Gene profiling of sample neurons is also conducted at multiple time points during the dopaminergic differentiation, evaluating a selected set of neuronal markers as well as markers of specific pathology related to neurodegeneration of PD (oxidative stress genes, mitochondrial genes, lysosomal genes, axon-guidance pathway genes). Alternatively, the global expression differences in these cell lines using commercially available microarray platforms for mRNAs to identify potential downstream targets or microRNAs can also be compared, since LRRK2 regulates miRNA-mediated translational repression (Gehrke et al. (2010) Nature 466:637-641). The availability of isogenic controls avoids confounding factors in data interpretation.

As the genetically corrected point mutation LRRK2 G2019S is expected to be phenotypically indistinguishable from a normal control cell line, it is ideal for studying disease phenotypes with only the causative mutation different from its ‘own’ control. With this approach, studying mutations can be directly assessed in a few isogenic case-control pairs without the need for large numbers of cases and controls that have to account for inter-individual biological differences and “noise.” This approach could be the basis of the use of single gene-defect cell lines and its isogenic corrected counterpart as a case-control pair to establish a set of tools for a disease-in-a-dish. Indeed, this new set of genetic-edited patient-specific cellular tools could be used to study as wide-variety of genetic disease and even their move common sporadic counterparts.

If there are no significant differences or frank pathology between the disease LRRK2 cells and corrected cells, these cells can be stressed with well-known toxicants and stressors to see if they are more prone to neurodegeneration as pathological signatures may be more easily detected if cells are challenged. Markers of vulnerability or oxidative stress above will be evaluated following challenge with neurotoxicants such as MPTP/MPP+ (Itano et al. (1994) Neurochem Int. 25:419-424; Viswanath et al. (2001) J. Neurosci. 21:9519-9528), a neurotoxicant specific for dopaminergic neurons; 6-hydroxydopamine to induce cell death and oxidative stress (Habisch et al. (2010) Cytotherapy 12:491-504; Kress et al. (2005) Neurobiol Dis. 20:639-645); and the pesticide rotenone, a mitochondrial toxin (Sherer et al. (2003) J. Neurosci. 23:10756-10764; Testa et al. (2005) Brain Res. Mol Brain Res 134:109-118).

All patents, patent applications and publications mentioned herein are hereby incorporated by reference in their entirety.

Although disclosure has been provided in some detail by way of illustration and example for the purposes of clarity of understanding, it will be apparent to those skilled in the art that various changes and modifications can be practiced without departing from the spirit or scope of the disclosure. Accordingly, the foregoing descriptions and examples should not be construed as limiting.

What is claimed is:

1. A method of modifying a G2019S mutant-encoding LRRK2 gene in a fibroblast or iPSC, the method comprising,
providing a fibroblast or iPSC that is heterozygous or homozygous for the LRRK2 G2019S mutation;
introducing at least one zinc finger nuclease (ZFN) that binds to and cleaves the mutant LRRK2 gene, thereby modifying the mutant LRRK2 gene.

2. The method of claim 1, wherein two ZFNs, that bind to first and second target sites and form a dimer upon binding, cleave the LRRK2 gene between the first and second target sites.

3. The method of claim 1, wherein cleavage results in modification of the gene sequence by non-homologous end joining or by homology directed-repair.

4. The method of claim 1, wherein cleavage results in integration of an exogenous sequence into the LRRK2 gene, and further wherein the method further comprises introducing the exogenous sequence into the cell.

5. The method of claim 1, wherein the cell is heterozygous for the modification.

6. The method of claim 1, wherein the cell is homozygous for the modification.

7. The method of claim 1, wherein the modification corrects the G2019S mutation.

8. The method of claim 1, wherein the modification introduces an additional mutation into the LRRK2 gene.

9. The method of claim 1, further comprising differentiating the ZFN-modified cells into dopaminergic neurons.

10. A fibroblast or iPSC produced by the method of claim 1.

11. The fibroblast or iPSC of claim 10, wherein the cell comprises at least two mutations in the LRRK2 gene.

12. An isolated culture of dopaminergic neurons generated by the method of claim 9.

13. The isolated culture of dopaminergic neurons according to claim 12, wherein the neurons comprise at least two mutations in the LRRK2 gene.


SEQUENCE LISTING (31 sequences)

SEQ ID NO: 1 - protein, 7 aa, Artificial Sequence
  Description of Artificial Sequence Synthetic peptide
QSGDLTR

SEQ ID NO: 2 - protein, 7 aa, Artificial Sequence
  Description of Artificial Sequence Synthetic peptide
RNDILAS

SEQ ID NO: 3 - protein, 7 aa, Artificial Sequence
  Description of Artificial Sequence Synthetic peptide
DRSNLSR

SEQ ID NO: 4 - protein, 7 aa, Artificial Sequence
  Description of Artificial Sequence Synthetic peptide
LRQDLKR

SEQ ID NO: 5 - protein, 7 aa, Artificial Sequence
  Description of Artificial Sequence Synthetic peptide
TSGNLTR

SEQ ID NO: 6 - protein, 7 aa, Artificial Sequence
  Description of Artificial Sequence Synthetic peptide
QSNQLRQ

SEQ ID NO: 7 - protein, 7 aa, Artificial Sequence
  Description of Artificial Sequence Synthetic peptide
QSGHLSR

SEQ ID NO: 8 - protein, 7 aa, Artificial Sequence
  Description of Artificial Sequence Synthetic peptide
RSDSLSV

SEQ ID NO: 9 - protein, 7 aa, Artificial Sequence
  Description of Artificial Sequence Synthetic peptide
QSSDLRR

SEQ ID NO: 10 - protein, 7 aa, Artificial Sequence
  Description of Artificial Sequence Synthetic peptide
RKDPLKE

SEQ ID NO: 11 - protein, 7 aa, Artificial Sequence
  Description of Artificial Sequence Synthetic peptide
RRDPLIN

SEQ ID NO: 12 - protein, 7 aa, Artificial Sequence
  Description of Artificial Sequence Synthetic peptide
QSGHLAR

SEQ ID NO: 13 - protein, 7 aa, Artificial Sequence
  Description of Artificial Sequence Synthetic peptide
RKWTLQG

SEQ ID NO: 14 - protein, 7 aa, Artificial Sequence
  Description of Artificial Sequence Synthetic peptide
QSSDLSR

SEQ ID NO: 15 - protein, 7 aa, Artificial Sequence
  Description of Artificial Sequence Synthetic peptide
QWSTRKR

SEQ ID NO: 16 - protein, 7 aa, Artificial Sequence
  Description of Artificial Sequence Synthetic peptide
RSDALTQ

SEQ ID NO: 17 - DNA, 28 bp, Artificial Sequence
  Description of Artificial Sequence Synthetic oligonucleotide
gcaaagattgctgactacggcattgctc

SEQ ID NO: 18 - DNA, 28 bp, Artificial Sequence
  Description of Artificial Sequence Synthetic oligonucleotide
tgatggcagcattgggatacagtgtgaa

SEQ ID NO: 19 - DNA, 998 bp, Homo sapiens
agctgagctaaacctctatgtggttttaggaaaatcaaaactattaaataaatggcaagtacaacaaaatcccatcaattcttatttaacatacttacattttgaaatagttaaaatattcatatgatcattgagagaattcagaattgcctttaagtaattgttcacatatacaaaagaaaagtctccaaaaattgggtctttgcctgagatagatttgtcttaaaattgaaatcattcacttatcagatttgacccttttttaaagcataactttgctgtgtaatattagacttatatgttttgatttccttctacaatatctcttaactttaagggacaaagtgagcacagaatttttgatgcttgacatagtggacatttatatttaaggaaattaggacaaaaattattataatgtaatcacatttgaataagatttcctgtgcattttctggcagatacctccactcagccatgattatataccgagacctgaaaccccacaatgtgctgcttttcacactgtatcccaatgctgccatcattgcaaagattgctgactacagcattgctcagtactgctgtagaatggggataaaaacatcagagggcacaccaggtaggtgatcaggtctgtctcataattctatcttcaggatggataaccactgacctcagatgtgagttcagaagagtcaaaaggaaaacagagtctatcacattgtgaacagaggtttattttgtgaaaaaatgcaagcatcacattgtgatttttatcattgtattttgtaggaaaaaaacaattgatgtaatttttcagggcaaaaactgaataaaaagaagagaatgtttgatatcaagttatatgttttaaagttagatttgtagattctttagatactctagaggtcataaaaagtaacagcaaaaactttagtctaggtattgttggcacttgtgaggcaaatcaaattcaggtccacaaattctttttc

SEQ ID NO: 20 - protein, 7 aa, Artificial Sequence
  Description of Artificial Sequence Synthetic peptide
RSDSLSA

SEQ ID NO: 21 - protein, 7 aa, Artificial Sequence
  Description of Artificial Sequence Synthetic peptide
QWGTRYR

SEQ ID NO: 22 - DNA, 28 bp, Artificial Sequence
  Description of Artificial Sequence Synthetic oligonucleotide
gcaaagattgctgactacagcattgctc

SEQ ID NO: 23 - DNA, 21 bp, Artificial Sequence
  Description of Artificial Sequence Synthetic primer
agggacaaagtgagcacagaa

SEQ ID NO: 24 - DNA, 22 bp, Artificial Sequence
  Description of Artificial Sequence Synthetic primer
gaggtcagtggttatccatcct

SEQ ID NO: 25 - DNA, 67 bp, Artificial Sequence
  Description of Artificial Sequence Synthetic oligonucleotide
cttttcacactgtatcccaatgctgccatcattgcaaagattgctgactacagcattgctcagtact

SEQ ID NO: 26 - DNA, 67 bp, Artificial Sequence
  Description of Artificial Sequence Synthetic oligonucleotide
agtactgagcaatgctgtagtcagcaatctttgcaatgatggcagcattgggatacagtgtgaaaag

SEQ ID NO: 27 - DNA, 67 bp, Artificial Sequence
  Description of Artificial Sequence Synthetic oligonucleotide
cttttcacactgtatcccaatgctgccatcattgcaaagattgctgactacggcattgctcagtact

SEQ ID NO: 28 - DNA, 67 bp, Artificial Sequence
  Description of Artificial Sequence Synthetic oligonucleotide
agtactgagcaatgccgtagtcagcaatctttgcaatgatggcagcattgggatacagtgtgaaaag

SEQ ID NO: 29 - DNA, 27 bp, Artificial Sequence
  Description of Artificial Sequence Synthetic primer
ctctttgcctgaaatgtccaactctag

SEQ ID NO: 30 - DNA, 29 bp, Artificial Sequence
  Description of Artificial Sequence Synthetic primer
caatcagcaagatgatgtatagccacctg

SEQ ID NO: 31 - protein, 9 aa, Artificial Sequence
  Description of Artificial Sequence Synthetic LAGLIDADG motif peptide
LAGLIDADG